(19) United States
(12) Patent Application Publication
(10) Pub. No.: US 2014/0236028 A1
(43) Pub. Date: Aug. 21, 2014

(54) BLOOD-PRESSURE SENSOR

(71) Applicant: Kabushiki Kaisha Toshiba, Minato-ku (JP)

(72) Inventors: Hiromi Yuasa, Kanagawa-ken (JP); Hideaki Fukuzawa, Kanagawa-ken (JP); Yoshihiko Fuji, Kanagawa-ken (JP); Alexander Devin Giddings, Kanagawa-ken (JP); Michiko Hara, Kanagawa-ken (JP); Shuichi Murakami, Tokyo (JP)

(73) Assignee: Kabushiki Kaisha Toshiba, Minato-ku (JP)

(21) Appl. No.: 14/261,917

(22) Filed: Apr. 25, 2014

Related U.S. Application Data
(63) Continuation of application No. 13/045,759, filed on Mar. 11, 2011.

(30) Foreign Application Priority Data
May 25, 2010 (JP) 2010-119568

(51) Int. Cl. A61B 5/021; A61B 5/00

(52) U.S. Cl. CPC A61B 5/02141; A61B 5/6833
USPC 600/485

(57) ABSTRACT

A blood-pressure sensor includes a substrate, a first electrode, a magnetization fixed layer, a nonmagnetic layer, a magnetization free layer, and a second electrode. The substrate is bent to generate a tensile stress at least in a first direction. The first electrode is provided on the substrate. The magnetization fixed layer has magnetization to be fixed in a second direction, and is provided on the substrate. The nonmagnetic layer is provided on the magnetization fixed layer. The magnetization free layer has a magnetization direction which is different from the first direction and from a direction perpendicular to the first direction. The second electrode is provided on the magnetization free layer.

### CROSS REFERENCE TO RELATED APPLICATION

This application is a continuation of U.S. application Ser. No. 13/045,759, filed Mar. 11, 2011, which is based upon and claims the benefit of priority from the prior Japanese Patent Application No. 2010-119568, filed on May 25, 2010, the entire contents of which are incorporated herein by reference.

### FIELD

Embodiments basically relate to a blood-pressure sensor.

### BACKGROUND

Sensing a blood pressure continuously without burden is required in a non-disease medical field while going about one's daily life. A small size blood-pressure sensor of bandaid type is needed in order to enable the continuous measurement of the blood-pressure with high accuracy.

There is known a blood pressure-sensor of cuff type. The sensor of cuff type applies a strong pressure on an arm or a finger to stop the blood flow thereof, thereby providing a blood-pressure measurement. For this reason, the continuous measurement is difficult to carry out. Moreover, it is difficult to miniaturize the blood-pressure sensor of cuff type, because the sensor needs a mechanism to apply such a strong pressure.

There is known a tonometry method as an enabling method to continuously measure a blood pressure. The tonometry method makes a sensor in contact with a human body to sense a strain due to an intra-arterial pressure of the body, thereby providing a blood-pressure measurement.

A device employing a MEMS (Micro Electro Mechanical System) pressure sensor is produced commercially on the basis of the tonometry method. The device is provided with a Si substrate having a thinned portion to strain in accordance with a change in an intra-arterial pressure. The strain of the thinned portion causes a resistance change to allow it to measure the blood pressure.

### BRIEF DESCRIPTION OF DRAWINGS

Aspects of this disclosure will become apparent upon reading the following detailed description and upon reference to accompanying drawings. The description and the associated drawings are provided to illustrate embodiments of the invention and not limited to the scope of the invention.

FIG. 1 is a view showing a use of a blood-pressure sensor 10 according to a first embodiment.

FIG. 2 is a view showing the blood-pressure sensor 10.

FIGS. 3A to 3C are views showing several arrangements of magnetization directions of a magnetization fixed layer, a magnetization free layer, and a direction of an applied tensile stress in a blood-pressure sensor according to the first embodiment.

FIG. 3D is a view showing the directions of the tensile stress and the magnetization of the magnetization free layer when a magnetostriction constant is positive or negative.

FIG. 3E is a graph showing dependence of magnetic anisotropy energy due to a magnetostriction effect on the angle between the magnetization direction of the magnetization free layer and the direction of a blood flow in a blood vessel.

FIG. 4 is a view showing conditions at the maximum and minimum blood pressures.

FIGS. 5A and 5B are views showing the directions of a blood flow and the magnetization of the magnetization free layer.

FIGS. 6A and 6B are views showing modifications of an MR element according to the first embodiment.

FIGS. 7A and 7B are views showing another two modifications of the MR element according to the first embodiment.

FIGS. 8A to 8D are views showing another four modifications of the MR element according to the first embodiment.

FIG. 9 is a view showing another modification of the MR element according to the first embodiment.

FIGS. 10A and 10B are views showing another two modifications of the MR element according to the first embodiment.

FIGS. 11A and 11B are views showing another two modifications of the MR element according to the first embodiment.

FIG. 12 is a view showing a blood-pressure sensor according to a second embodiment.

FIG. 13 is a view to explain an operation principle of the blood-pressure sensor according to the second embodiment.

FIG. 14 is a view showing a modification of the blood-pressure sensor according to the second embodiment.

FIGS. 15A and 15B are views showing another two modifications of the blood-pressure sensor according to the second embodiment.

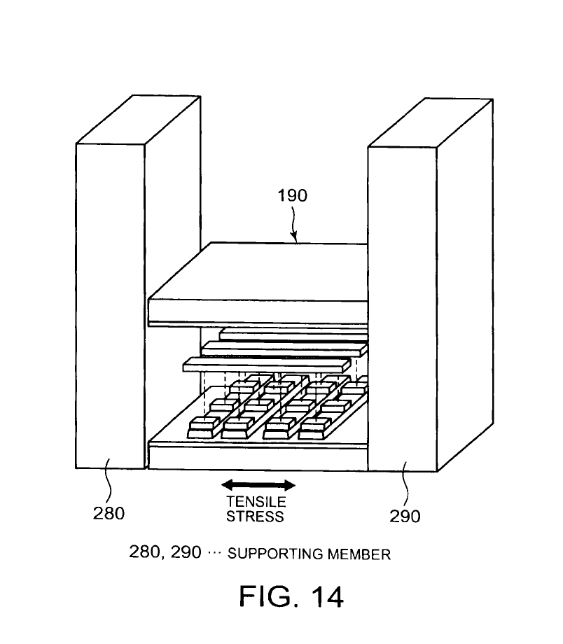

FIG. 14 is a view showing another modification of the blood-pressure sensor according to the second embodiment.

FIGS. 17A to 17B are views showing another three modifications of the blood-pressure sensor according to the second embodiment.

FIG. 18 is a view showing another modification of the blood-pressure sensor according to the second embodiment.

FIG. 19 is a view showing another modification of the blood-pressure sensor according to the second embodiment.

FIG. 20 is a view showing another modification of the blood-pressure sensor according to the second embodiment.

FIG. 21 is a view showing another modification of the blood-pressure sensor according to the second embodiment.

FIGS. 22A and 22B are views showing blood-pressure sensors according to a third embodiment.

FIGS. 23A to 23C are views showing another modification of the blood-pressure sensor according to the third embodiment.

FIG. 24 is a view showing a blood-pressure sensor according to a fourth embodiment.

FIG. 25 is a view showing a graph for resistance measurements and magnetization arrangements of a processed MR element.

FIG. 26 is a view showing a graph for resistance measurements of a MR element to which a tensile stress is applied, and magnetization arrangements of the MR element with and without the tensile stress.

FIG. 27 is a view to explain how to strain a prepared MR element.

FIG. 28 is a view showing a blood-pressure measurement system according to a fifth embodiment.

FIG. 29 is a flow chart to illustrate operation steps of the blood-pressure measurement system according to the fifth embodiment.

FIG. 30 is a view showing a method to measure a resistance change amount of a blood-pressure sensor.

FIG. 31 is a view to illustrate a selecting method of an MR element having a maximum absolute value of the resistance change amount involved in coarctation and vascular dilation.

FIG. 32 is a view to explain how the blood-pressure measurement system according to the fifth embodiment performs a transformation between resistance values and blood pressures.

### DESCRIPTION

As will be described below, according to an embodiment, a blood-pressure sensor includes a substrate, a first electrode, a magnetization fixed layer, a nonmagnetic layer, a magnetization free layer, and a second electrode. The substrate is bent to generate a tensile stress at least in a first direction. The first electrode is provided on the substrate. The magnetization fixed layer has magnetization to be fixed in a second direction, and is provided on the substrate. The nonmagnetic layer is provided on the magnetization fixed layer. The magnetization free layer has a magnetization direction which is different from the first direction and from a direction perpendicular to the first direction. The second electrode is provided on the magnetization free layer.

According to another embodiment, a blood-pressure sensor includes a first substrate, a pair of a first supporting member and a second supporting member, a magnetoresistive element, a second substrate, a third supporting member, and an elastic body. The first substrate is bent to generate a tensile stress at least in a first direction. The pair of the first supporting member and the second supporting member is provided on the first substrate, and is separated from each other. The second substrate is provided so that the magnetoresistive element is sandwiched between the first substrate and the second substrate. The third supporting member connects the first supporting member and the second supporting member. The elastic body is provided between the second substrate and the third supporting member. The magnetoresistive element includes two or more first electrodes, a magnetization fixed layer, a nonmagnetic layer, a magnetization free layer, and a second electrode. The first electrodes are provided between the first supporting member and the second supporting member, and are provided on the first substrate. The magnetization fixed layer has magnetization to be fixed in a second direction, and is provided on the first substrate. The nonmagnetic layer is provided on the magnetization fixed layer. The magnetization free layer has magnetization whose direction is variable, and is provided on the nonmagnetic layer. The second electrode is provided on the magnetization free layer. In addition, the magnetization direction of the magnetization free layer is different from the first direction in which the tensile stress is generated and from a direction perpendicular to the first direction.

According to another embodiment, a blood-pressure sensor includes a first substrate, a pair of a first supporting member and a second supporting member, a magnetoresistive element, a second substrate, and a housing. The first substrate is bent to generate a tensile stress at least in a first direction. The pair of the first supporting member and the second supporting member is provided on the first substrate, and the first supporting member and the second supporting member are separated from each other. The second substrate connects the first supporting member and the second supporting member, and is provided so that the magnetoresistive element is sandwiched between the first substrate and the second substrate. The housing has a constant pressure therein, and is provided on the second substrate. The magnetoresistive element includes two or more first electrodes, a magnetization fixed layer, a nonmagnetic layer, a magnetization free layer, and a second electrode. The first electrodes are provided on the first substrate, and between the first supporting member and the second supporting member. The magnetization fixed layer has magnetization to be fixed in a second direction, and is provided on the first substrate. The nonmagnetic layer is provided on the magnetization fixed layer. The magnetization free layer has magnetization whose direction is variable, and is provided on the nonmagnetic layer. The second electrode is provided on the magnetization free layer. In addition, the magnetization direction of the magnetization free layer is different from the first direction in which the tensile stress is generated, and from a direction perpendicular to the third direction.

According to another embodiment, a blood-pressure sensor includes a substrate, a first interconnection, a magnetoresistive element, and a second interconnection. The substrate is bent to generate a tensile stress at least in a first direction. The first interconnection is provided in a column direction and on the substrate. The second interconnection is provided in a row direction so that the magnetoresistive element is sandwiched between the first interconnection and the second interconnection. The magnetoresistive element includes a first electrode, a magnetization fixed layer, a nonmagnetic layer, a magnetization free layer, and a second electrode. The first electrode is provided on the first interconnection. The magnetization fixed layer has magnetization to be fixed in a second direction and is provided on the first electrode. The nonmagnetic layer is provided on the magnetization fixed layer. The magnetization free layer has magnetization whose direction is variable and is provided on the nonmagnetic layer. The second electrode is provided on the magnetization free layer. In addition, the magnetization direction of the magnetization free layer is different from the first direction in which the tensile stress is generated, and from a direction perpendicular to the third direction.

Embodiments will be described below with reference to drawings. The drawings are conceptual. Therefore, a relationship between the thickness and width of each portion or a proportionality factor among the respective portions are not necessarily the same as an actual one. Even when the same portions are drawn, their sizes or proportionality factors may be represented differently from each other. Wherever possible, the same reference numerals or marks will be used to denote the same portions or the like throughout the drawings, and overlapped descriptions will be omitted.

### First Embodiment

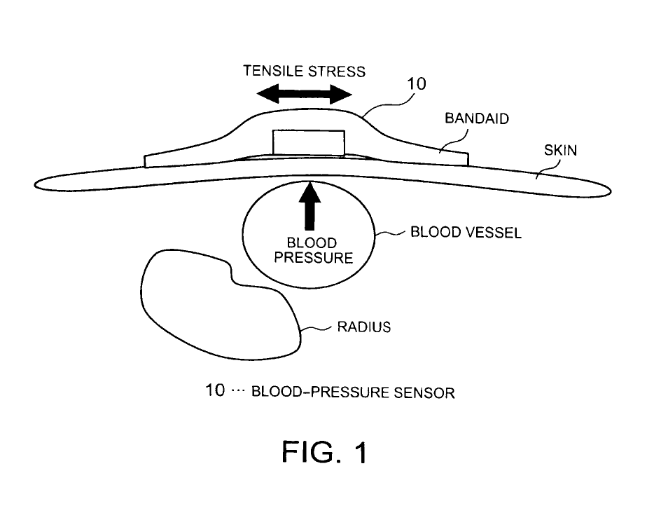

FIG. 1 is a view showing a blood-pressure sensor 10 according to a first embodiment and a blood-pressure measurement site.

The blood-pressure sensor 10 is stuck to the blood-pressure measurement site, and is, therefore, formed in a bandaid shape or the like to adhere to a skin surface. That is, the blood-pressure sensor 10 is arranged so that the blood-pressure sensor 10 is in contact with a skin beneath which an arterial vessel leads. The direction of the blood flow is perpendicular to the plane of paper, and is meant by a longitudinal direction of the blood vessel. If there is no arterial vessel near a skin surface, it is difficult to measure the blood pressure near the skin surface. The following body sites allow it to sense pulsations from a body surface and beneath the body surface:
Medial biceps brachii muscles groove (humeral artery);
lateral lower end of forehand (radial artery) between tendons of flexor carpi radialis and brachioradial muscle;
medial lower end of forehand between flexor carpi ulnaris muscle tendon and superficial digital flexor tendon (ulnar artery);
ulnar side of extensor pollicis longus tendon (first dorsal metacarpal artery);
axillary cavity (arteria axillaries);
triangular part (crural artery);
lower portion of anterior surface of leg and outside of tendon of tibialis anterior (anterior tibial artery);
lower back portion of malleolus medialis (arteria tibialis posterior);
outside of extensor pollicis longus tendon (arteria dorsalis pedis);
triangle of arteria carotis (arteria carotis communis);
front portion of masseter arrest site (arteria facialis);
behind sternocleidomastoideus arrest site and between the arrest site and beginning of cowl muscle (arteria occipitalis); and
front of external acoustic opening (arteria temporalis superficialis).
   Accordingly, the blood-pressure sensor 10 can be arranged on the above-mentioned body sites. That is, these body sites correspond to the blood-pressure measurement sites. The blood-pressure sensor 10 is stuck onto the skin surfaces having these body sites beneath the skin surfaces.

As shown in FIG. 1, a blood vessel expands radially to upheave the skin above the blood vessel, thereby producing a blood pressure. Then, the skin is subjected to a tensile stress in a direction perpendicular to the direction in which the blood pressure acts. At the same time, the blood-pressure sensor 10 is subjected to a tensile stress in a certain direction (a first direction).

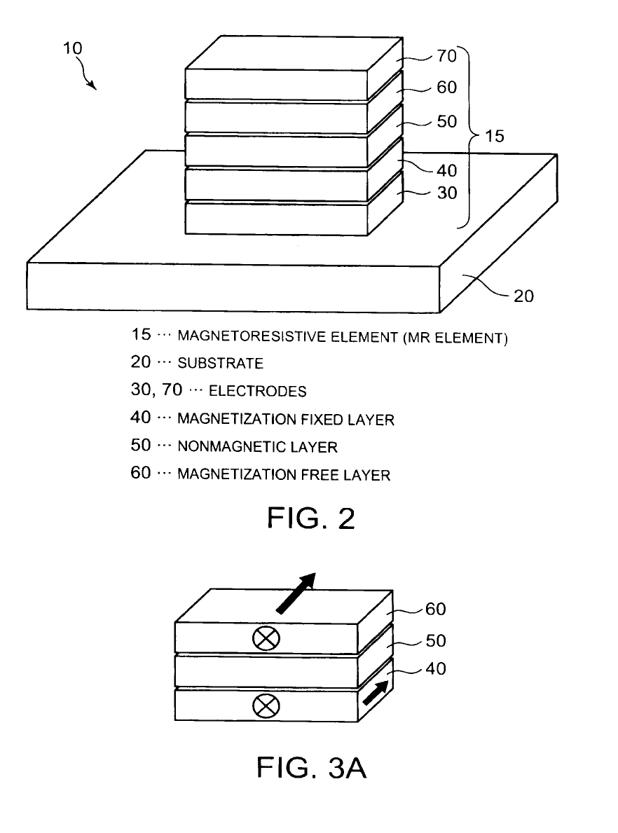

FIG. 2 is a view showing the blood-pressure sensor 10.

The blood-pressure sensor 10 is provided with an electrode 30 on a substrate 20 and a magnetization fixed layer 40 on the electrode 30. The magnetization of the magnetization fixed layer 40 is fixed in one direction. The blood-pressure sensor 10 is provided further with a nonmagnetic layer 50 on the magnetization fixed layer 40 and a magnetization free layer 60 on the nonmagnetic layer 50. The magnetization direction of the magnetization free layer 60 is controllably variable. An electrode 70 is provided onto the magnetization free layer 60. Alternatively, the magnetization fixed layer 40 and the magnetization free layer 60 may replace each other for the arrangement thereof. The magnetization fixed layer 40 and the magnetization free layer 60 are ferromagnetic. A structure including the electrode 30, the magnetization fixed layer 40, the nonmagnetic layer 50, the magnetization free layer 60, and the electrode 70 is called a magnetoresistive element (referred to as an “MR element” below) 15. Another structure excluding the electrodes 30 and 70 from the MR element 15 is called an MR film. Alternatively, an insulating layer including, e.g., aluminum oxide may be provided between the substrate 20 and the electrode 30.

Materials for the substrate 20 include an insulator or a semiconductor. Examples of the insulator include polyimide, i.e., a plastic material. Examples of the semiconductor include silicon.

The magnetization fixed layer 40 is ferromagnetic. Materials for the magnetization fixed layer 40 include an FeCo alloy, a CoFeB alloy, and a NiFe alloy. The thickness of the magnetization fixed layer 40 ranges from 2 nm to 6 nm, for example.

Metals or insulators can be employed for the nonmagnetic layer 50. The metals include Cu, Au, and Ag, for example. When employing the metals for the nonmagnetic layer 50, the thickness thereof ranges from 1 nm to 7 nm, for example. The insulators include magnesium oxides (MgO), aluminum oxides (Al₂O₃), titanium oxides (TiO), and zinc oxides (ZnO), for example. When employing the insulators for the nonmagnetic layer 50, the thickness thereof ranges from 0.6 nm to 2.5 nm, for example.

The magnetization free layer 60 is ferromagnetic. Materials for the magnetization free layer 60 include a FeCo alloy and a NiFe alloy, for example. The materials will also include a Fe—Co—Si-B alloy, a Tb-M-Fe alloy having λs>100 ppm (examples of M include Sm, Eu, Gd, Dy, Ho and Er), a Tb—M1-Fe-M2 alloy (examples of M1 include Ti, Cr, Mn, Co, Cu, Nb, Mo, W and Ta; examples of M2 include Ti, Cr, Mn, Co, Cu, Nb, Mo, W and Ta), a Fe-M3-M4-B alloy (examples of M3 include Ti, Cr, Mn, Co, Cu, Nb, Mo, W and Ta; examples of M4 include Ce, Pr, Nd, Sm, Tb, Dy and Er), Ni, Al—Fe, a ferrite (Fe₃O₄, (FeCo)₃O₄). The thickness of the magnetization free layer 60 is not less than 2 nm, for example.

Alternatively, the magnetization free layer 60 may be a double layer. A layer including one of the materials to be mentioned below is laminated on a FeCo alloy layer to form the double layer. The materials include a Fe—Co—SiB alloy, a Tb—M—Fe alloy having λs>100 ppm (examples of M include Sm, Eu, Gd, Dy, Ho and Er), a Tb-M1-Fe-M2 alloy (examples of M1 include Ti, Cr, Mn, Co, Cu, Nb, Mo, W and Ta; examples of M2 include Ti, Cr, Mn, Co, Cu, Nb, Mo, W and Ta), a Fe-M3-M4-B alloy (examples of M3 include Ti, Cr, Mn, Co, Cu, Nb, Mo, W and Ta; examples of M4 include Ce, Pr, Nd, Sm, Tb, Dy and Er), Ni, Al—Fe, a ferrite (Fe₃O₄, (FeCo)₃O₄).

Nonmagnetic Au, Cu, Ta, Al, etc. can be employed for the electrodes 30 and 70, for example. Soft magnetic materials are also employed for the electrodes 30 and 70, thereby allowing it to reduce magnetic noises which are caused by an external field and influence the MR element 15. The soft magnetic materials include a permalloy (NiFe alloy) and an FeSi alloy, for example. The MR element 15 is covered with insulators (not shown), such as aluminum oxide (e.g., Al₂O₃) and silicon oxide (e.g., SiO₂), so that the electrodes 30 and 70 do not short out.

An operation principle of the blood-pressure sensor 10 will be described below.

The blood-pressure sensor 10 operates on the basis of an “inverse magnetostriction effect” and the “MR effect”. The inverse magnetostriction effect is possessed by a ferromagnetic material, and the MR effect comes from the multilayer of the magnetization fixed layer 40, the nonmagnetic layer 50, and the magnetization free layer 60.

The “inverse magnetostriction effect” and the “MR effect” due to a portion of the blood-pressure sensor 10 will be explained below. A current is passed through the electrode 30 and the electrode 70 in a direction perpendicular to the lamination direction of the magnetization fixed layer 40, the nonmagnetic layer 50, and the magnetization free layer 60 to read out a resistance change due to a change in a relative angle between the magnetization directions of the two layers 40, 50, thereby allowing it to obtain the “MR effect.” When the magnetization direction of the magnetization free layer 60 and the direction of the tensile stress are different from each other, the inverse magnetostriction effect induces the MR effect. A resistance change amount due to the MR effect is referred to as an “MR change amount” and the amount divided by the resistance is referred to as an “MR change rate.”

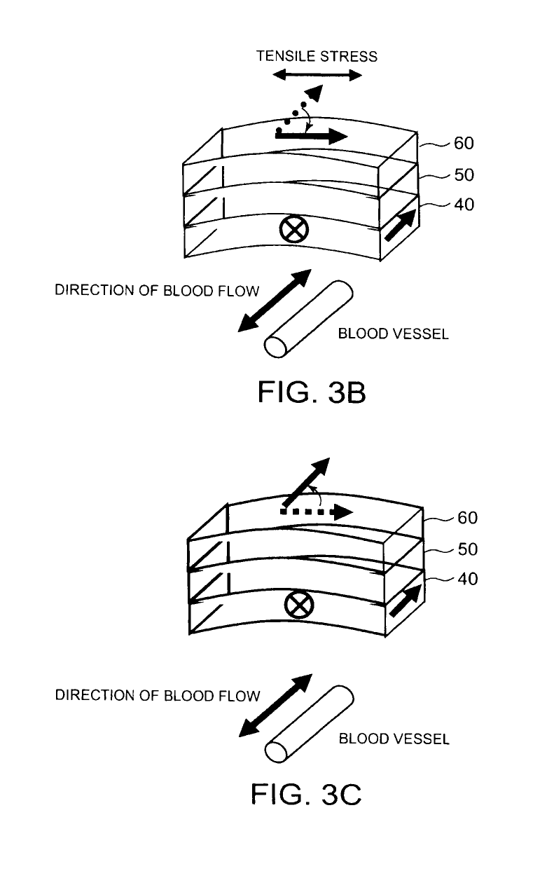

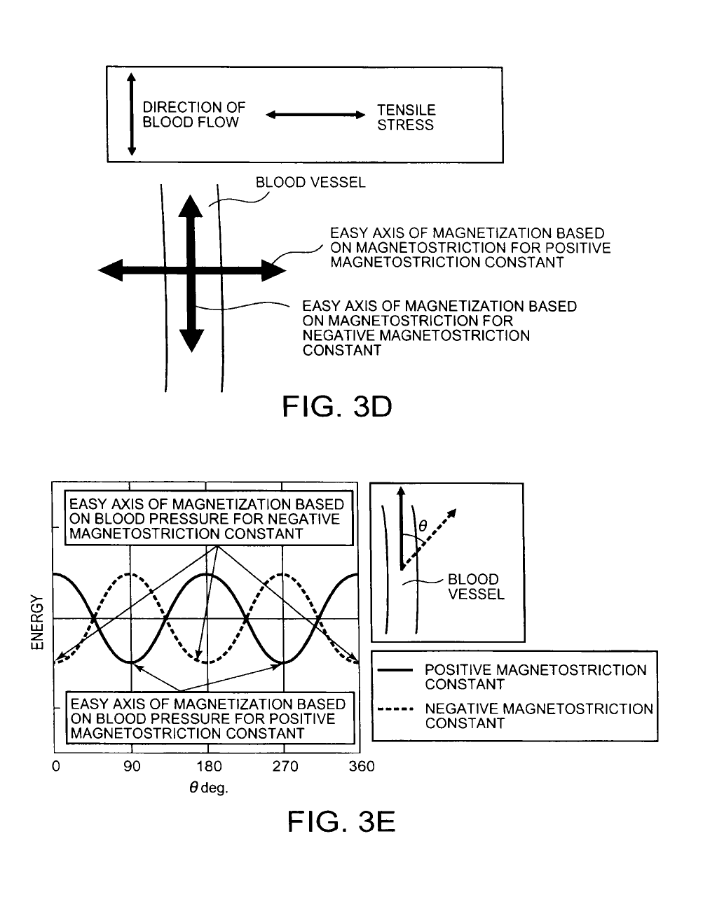

FIGS. 3A to 3C are views showing several arrangements of the magnetization directions of the magnetization fixed layer 40, the magnetization free layer 60, and the direction of the tensile stress. The views also show relationships therebetween. FIGS. 3A to 3C include the magnetization fixed layer 40, the nonmagnetic layer 50, and the magnetization free layer 60. FIG. 3D is a view showing the directions of the tensile stress and the magnetization of the magnetization free layer 60 when a magnetostriction constant is positive or negative. FIG. 3E is a graph showing the angle dependence of magnetic anisotropy energy due to an inverse magnetostriction effect. The magnetic anisotropy energy depends on the angle between the magnetization direction of the magnetization free layer 60 and the direction of a blood flow in a blood vessel.

FIG. 3A is a view showing an MR film without any tensile stress applied. The magnetization of the magnetization fixed layer 40 has the same direction as that of the magnetization free layer 60.

FIG. 3B is a view showing the MR film to which a tensile stress is applied. FIG. 3B shows also the direction of a blood flow. The directions of the blood flow and the tensile stress are at right angles to each other. The tensile stress is applied in a direction perpendicular to the magnetization directions of the magnetization fixed layer 40 and the magnetization free layer 60. At this time, the magnetization of the magnetization free layer 60 rotates so that the magnetization thereof is in the same direction as the tensile stress. This is called the “inverse magnetostriction effect.” Furthermore, the magnetization of the magnetization fixed layer 40 is fixed in one direction. Therefore, the magnetization of the magnetization free layer 60 rotates to change the relative angle between the magnetization directions of the magnetization fixed layer 40 and the magnetization free layer 60. The magnetization direction of the magnetization fixed layer 40 is illustrated as an example, and is not necessarily the same as shown in FIG. 3B.

The inverse magnetostriction effect changes the direction of an easy axis of magnetization in accordance with plus or minus of a magnetostriction constant of a ferromagnetic material. Most materials showing large inverse magnetostriction effects have a positive magnetostriction constant. The positive magnetostriction constant makes the easy axis of magnetization in the direction of action of the tensile stress. That is, the magnetization of the magnetization free layer 60 will rotate in the direction of the easy axis of magnetization.

Therefore, the positive magnetostriction constant of the magnetization free layer 60 requires the magnetization direction thereof to be preliminarily set in a direction different from a direction of action of the tensile stress.

The negative magnetostriction constant of the magnetization free layer 60 makes the easy axis of magnetization in a direction perpendicular to the direction of action of the tensile stress. This condition is shown in FIG. 3C. The negative magnetostriction constant of the magnetization free layer 60 requires the magnetization direction thereof to be preliminarily set in a direction different from a direction perpendicular to the direction of action of the tensile stress. The magnetization direction of the magnetization fixed layer 40 is illustrated as an example, and is not necessarily the same as shown in FIG. 3C.

FIG. 3D is a view showing the above-mentioned directions for the positive and negative magnetostriction constants. The easy axis of magnetization is in a direction parallel or perpendicular to the direction of a blood flow.

FIG. 3E is a graph showing dependence of magnetic anisotropy energy due to the inverse magnetostriction effect on the angle between the magnetization direction of the magnetization free layer 60 and the direction of the blood flow in a blood vessel. The horizontal axis represents the angle θ(°) between the magnetization direction of the magnetization free layer 60 and the direction of the blood flow. The vertical axis represents the magnetic anisotropy energy due to the inverse magnetostriction effect by an arbitrary unit. The angle θ at the minimum of the magnetic anisotropy energy corresponds to the easy axis of magnetization. The angle θ at the maximum of the magnetic anisotropy energy corresponds to the direction of a hard axis of magnetization. The hard axis of magnetization means a direction in which it is hard to rotate the magnetization of the magnetization free layer 60.

The MR change amount is defined as a relative change in the resistance of the MR film due to a change in the angle between the magnetization directions of the magnetization fixed layer 40 and the magnetization free layer 60.

The larger the amount of change in the angle between the magnetization directions of the magnetization free layer 60 and the magnetization fixed layer 40, the larger the MR change amount. Therefore, the magnetization free layer 60 makes the magnetization thereof align in the direction of the hard axis of magnetization without any tensile stress applied to maximize the MR change amount.

The magnetization of the magnetization free layer 60 rotates in a clockwise direction or in a counterclockwise direction at the maximum. The probability of rotating in the counterclockwise direction is comparable to that of rotating in the clockwise direction. In this case, the MR change amount will take two values substantially. For this reason, the magnetization of the magnetization free layer 60 is preliminarily set to deviate slightly from the direction of the hard axis of magnetization. When the magnetostriction constant of the magnetization free layer 60 is positive, the magnetization direction of the magnetization free layer 60 is made not to be parallel to the blood-flow direction. When the magnetostriction constant of the magnetization free layer 60 is negative, the magnetization direction of the magnetization free layer 60 is made not to be perpendicular to the blood-flow direction.

That is, when no tensile stress is applied, the magnetization direction of the magnetization free layer 60 is made not to be parallel to both the directions of the easy and hard axes of magnetization. It is, therefore, necessary to weakly fix the magnetization of the magnetization free layer 60 so that the magnetization direction thereof does not become perpendicular or parallel to the blood-flow direction independently of plus or minus of the magnetostriction constant thereof.

When the magnetostriction constant of the magnetization free layer 60 is positive, changing θ shown in FIG. 3E from 10° to 45°, 135° to 170°, 190° to 225° or 315° to 350° allows it to increase the MR change amount as a result of the increased amount of magnetization rotations. When the magnetostriction constant of the magnetization free layer 60 is negative, changing θ shown in FIG. 3E from 45° to 80°, 100° to 135°, 225° to 260° or 280° to 315° allows it to increase the MR change amount as a result of increasing the amount of the magnetization rotation.

The pressure to be experienced by the blood-pressure sensor 10 from a blood vessel changes in accordance with the respective conditions at maximum and minimum blood pressures involved in the pulsation when measuring a blood pressure using the blood-pressure sensor 10. At the maximum blood pressure, a tensile stress acts strongly on the skin surface. At the minimum blood pressure, the tensile stress acts weakly on the skin surface. The stronger and weaker tensile stresses correspond to a periodic oscillation of the pulsation.

The difference in height of the blood pressure involved in the periodic oscillation of the pulsation allows it to judge whether or not the blood-pressure sensor 10 can actually measure the blood pressure. On that basis, the blood-pressure sensor 10 or the controller involved therein calculates the magnitudes of the maximum blood pressure and the minimum one.

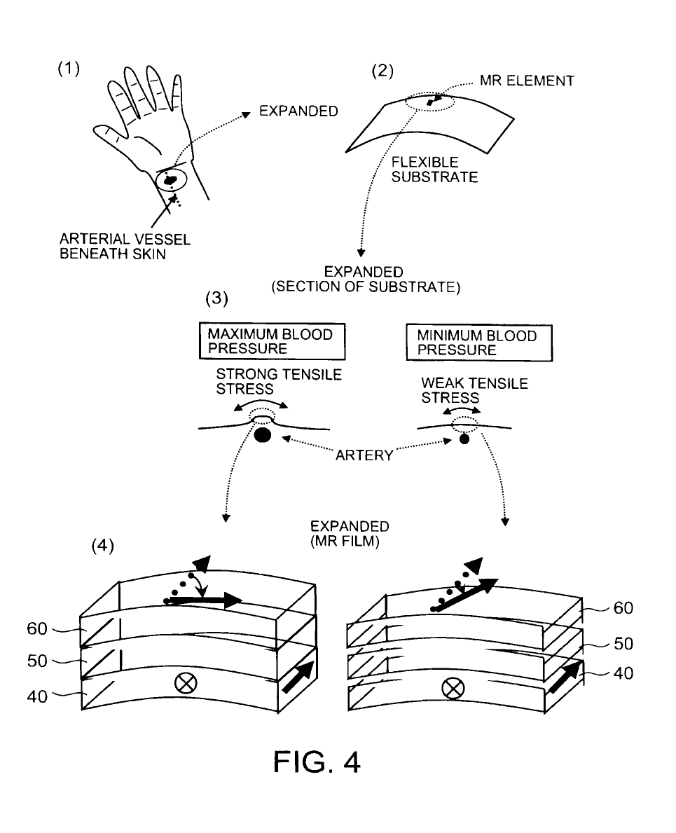

FIG. 4 is a view showing conditions at the maximum and minimum blood pressures. FIG. 4 illustrates an example when sticking the blood-pressure sensor 10 on a wrist. As shown in the part (1) of FIG. 4, the blood-pressure sensor 10 formed on a substrate is arranged just above an arterial blood vessel.

In the part (2) of FIG. 4, the MR element is arranged on the substrate (to be flexible in FIG. 4). The substrate is bent as it follows the outer diameter of the arterial blood vessel. The tensile stress acts substantially perpendicularly to the blood-flow direction.

In the part (3) of FIG. 4, the substrate and the arterial blood vessel are viewed from the blood flow direction at the maximum and minimum blood pressures. The maximum blood pressure fully swells the arterial blood vessel to increase the tensile stress acting on the substrate. The minimum blood pressure less swells the arterial blood vessel to decrease the tensile stress acting on the substrate.

The part (4) of FIG. 4 shows an arrangement for the MR element to sense the maximum and minimum blood pressures. The explanation will be made below for the positive magnetostriction constant. When no blood pressure is applied, the magnetization of the magnetization free layer 60 is aligned in a direction other than the direction in which the tensile stress acts. When the maximum blood pressure is applied, the substrate strains much to rotate the magnetization of the magnetization free layer 60 largely. The minimum blood pressure makes the substrate strain less than the maximum blood pressure, thereby rotating the magnetization of the magnetization free layer 60 in an intermediate direction between the initial direction and the direction at the maximum blood pressure.

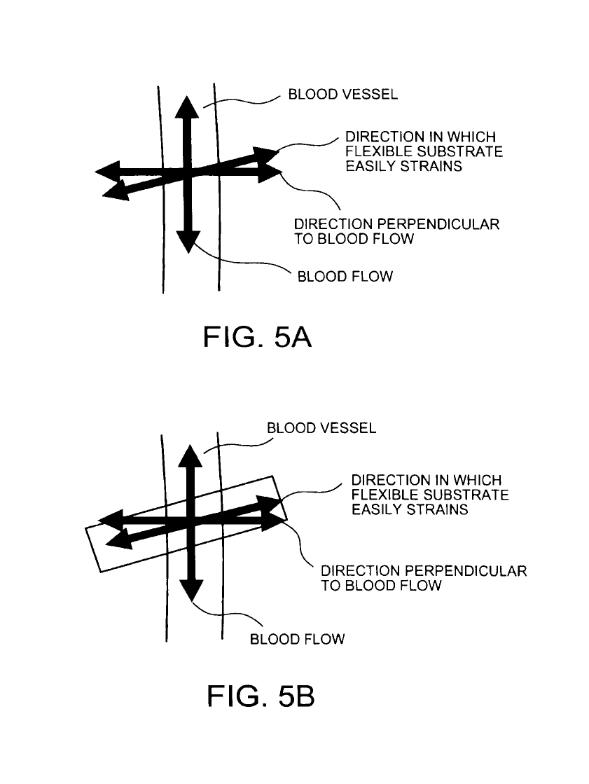

There may be cases in which a blood vessel cannot be clearly determined. Such cases include measuring the blood pressure at arteria occipitalis. It is difficult to clearly determine a blood vessel even near arteria radialis of a wrist. In contrast, if the flexible substrate of the blood-pressure sensor strains anisotropically, such cases do not occur. Specifically, when a tensile stress is applied to a skin, the substrate stuck on the skin is provided with a tensile character to give a prescribed specific tensile direction to the substrate, thereby setting up the specific tensile direction and the magnetization direction of the magnetization free layer 60. A conceptual view thereof is shown in FIG. 5A. FIGS. 5A and 5B are views showing the directions of a blood flow and the magnetization of the magnetization free layer. The specific method to give the strain anisotropy is making the flexible substrate a rectangular or ellipsoidal shape having a long axis and a short axis. A conceptual view for the method is shown in FIG. 5B. When the substrate is ellipsoidal in shape, the substrate easily strains in the long axis direction (i.e., the longitudinal direction). When the substrate is rectangular in shape, the substrate easily strains in the long side direction (i.e., the longitudinal direction). The longitudinal direction preferably intersects with a blood flow direction.

The metallic and insulating nonmagnetic layers 50 bring about a GMR (Giant magnetoresistance) effect and a TMR (Tunnel magnetoresistance) effect, respectively. The first embodiment and a second embodiment to be described below employ a CPP (Current perpendicular to plane)-GMR effect by passing a current through a laminated layer perpendicularly to the lamination direction thereof. The current is passed through the electrode 30 and the electrode 70. When employing the TMR effect, the current is passed therethrough as well as in the GMR effect.

When measuring a blood pressure, a change in the blood pressure is derived from a correlation between accumulated data on the blood pressures of test subjects and MR change rates corresponding thereto. This will be described below.

### Modification 1

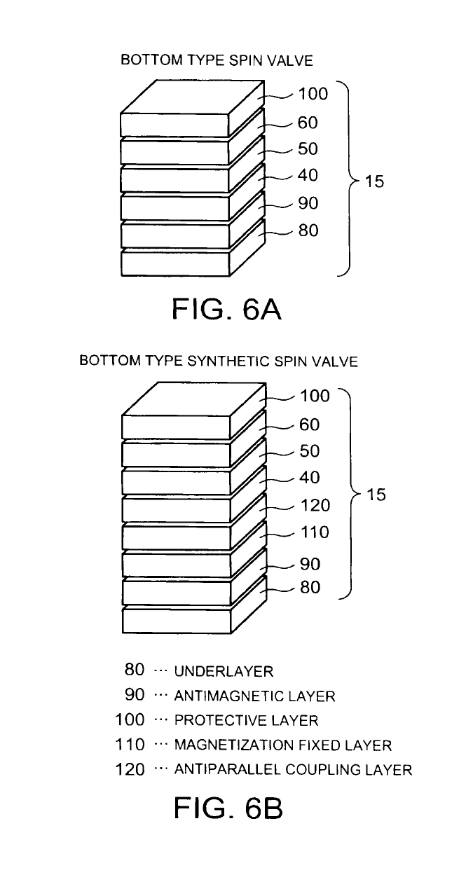

FIGS. 6A and 6B are views showing modifications of the MR element 15 according to the first embodiment. The electrodes are not shown. Descritions about the same compositions as those in the first embodiment will be omitted.

A modified MR element 15 shown in FIG. 6A is provided with an underlayer 80, an antiferromagnetic layer 90, the magnetization fixed layer 40, the nonmagnetic layer 50, the magnetization free layer 60, and a protective layer 100 which are laminated in this order from the bottom. This structure is called a bottom type spin valve film.

The underlayer 80 enhances a crystal orientation of the spin valve film laminated thereon. Materials of the underlayer 80 include amorphous Ta matching the substrate easily, Ru, NiFe, and Cu enhancing the crystal orientations of the upper layers formed thereon. The lamination of the amorphous Ta and one of crystalline Ru, NiFe or Cu can strike a balance between wettability and crystal orientations. The thickness of the underlayer 80 ranges from 0.5 nm to 5 nm, for example.

The protective layer 100 protects the MR element 15 from damages on manufacturing the MR element 15. Materials of the protective layer 100 include Cu, Ta, Ru, for example. The thickness of the protective layer 100 ranges from 1 nm to 20 nm, for example.

The MR element 15 shown in FIG. 6B is provided with the underlayer 80, an antiferromagnetic layer 90, a magnetization fixed layer 110, an antiparallel coupling layer 120, the magnetization fixed layer 40, the nonmagnetic layer 50, the magnetization free layer 60, and the protective layer 100 in this order from the substrate. This laminated structure is called a “bottom type synthetic spin valve film”, and enables it to increase a fixing strength for the magnetization of the magnetization fixed layer 40.

Exchange coupling due to the antiferromagnetic layer 90 fixes the magnetization of the magnetization fixed layer 110 in one direction. The material employed for the magnetization fixed layer 110 is the same as that for the magnetization fixed layer 40. The magnetization fixed layer 110 is made to have the thickness which is mostly the same as a magnetic thickness (the product of the saturation magnetization “Bs” and the film thickness “t”, Bs×t) of the magnetization fixed layer 40. For example, the thickness of the magnetization fixed layer 110 ranges from 2 nm to 6 nm.

The antiparallel coupling layer 120 couples the magnetization fixed layer 40 and the magnetization fixed layer 110 with each other so that the magnetization of the magnetization fixed layer 40 and the magnetization of the magnetization fixed layer 110 are antiparallel to each other. Therefore, even if the exchange coupling energy from the antiferromagnetic layer 90 is constant, the fixing magnetic field for the magnetization of the magnetization fixed layer 40 can be increased. Therefore, influences of magnetic noises generated from electronic devices can be reduced. Materials of the antiparallel coupling layer 120 include Ru and Ir, for example. The thickness of the antiparallel coupling layer 120 ranges from 0.8 nm to 1 nm, for example.

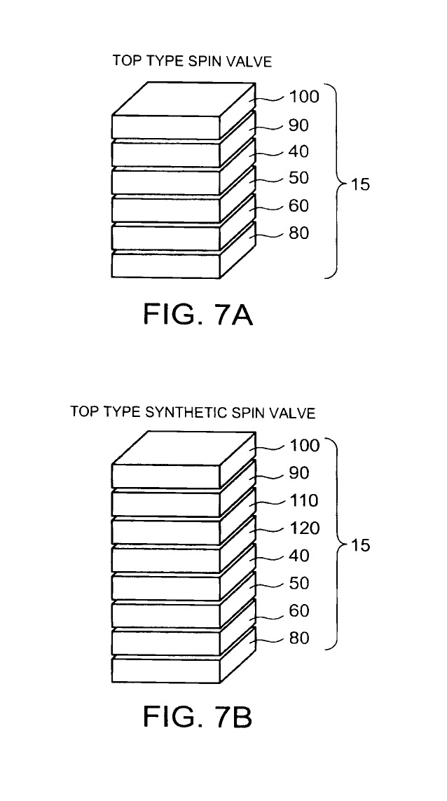

FIGS. 7A and 7B are views also showing modifications of the MR element 15 according to the first embodiment. As shown in FIG. 7A, the MR element 15 of this modification can also be made as a “top type spin valve film” with the magnetization free layer 60, the nonmagnetic layer 50, the magnetization fixed layer 40, the antiferromagnetic layer 90, and the protective layer 100 laminated in this order on the underlayer 80.

As shown in FIG. 7B, the MR element 15 of this modification can also be made as a “top type synthetic spin valve film” with the magnetization free layer 60, the nonmagnetic layer 50, the magnetization fixed layer 40, the antiparallel coupling layer 120, the magnetization fixed layer 110, the antiferromagnetic layer 90, and the protective layer 100 laminated in this order on the underlayer 80. The layers included in the top type spin valve film and the top type synthetic spin valve film are the same as those included in the bottom type spin valve film and the bottom type synthetic spin valve film, descriptions thereon being omitted.

A method to make the magnetization of the magnetization free layer 60 in a direction different from the direction of the tensile stress employs interlayer coupling between the magnetization of the magnetization fixed layer 40 and the magnetization of the magnetization free layer 60 via the nonmagnetic layer 50. The metallic nonmagnetic layer 50 with a thickness of 3 nm or less brings about the interlayer coupling so that both the magnetization directions are parallel to each other as well as the insulating nonmagnetic layer 50 with a thickness of 1.5 nm or less. Fixing the magnetization of the magnetization free layer 40 in the direction different from the direction of the tensile stress allows it to make the magnetization of the magnetization free layer 60 in the direction different therefrom with low energy.

Moreover, the magnetization free layer 60 is deposited by sputtering while applying a magnetic field thereto, thereby allowing it to fix the magnetization of the magnetization free layer 60 in one direction. The magnetization easily is set in the direction of the magnetic field during the deposition. It is, therefore, preferable to deposit a film for the magnetization free layer 60 while applying a magnetic field thereto.

### Modification 2

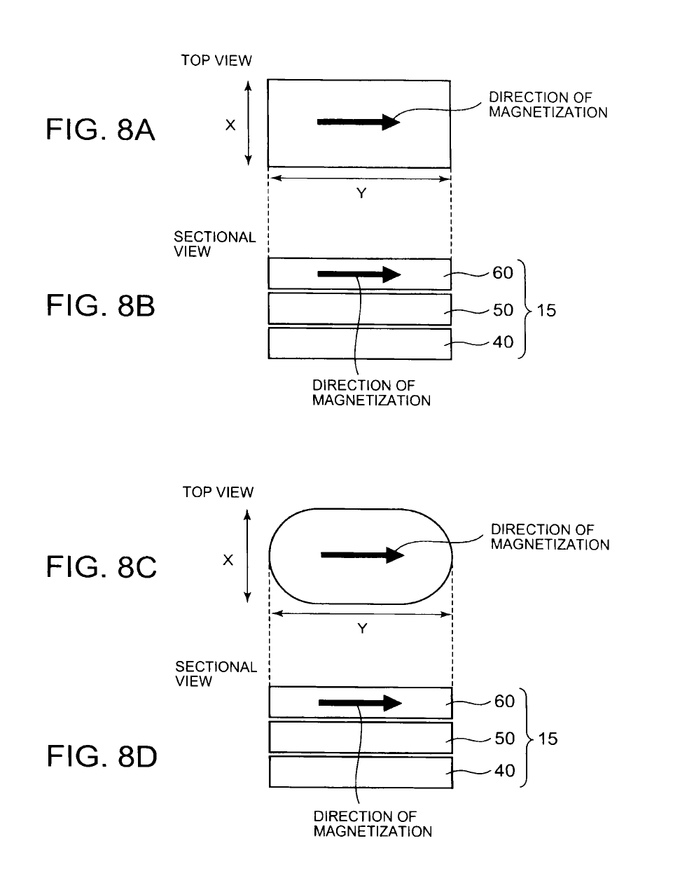

FIGS. 8A to 8D are views showing modifications of the MR element 15 according to the first embodiment. The electrodes are not shown in FIG. 8. Descriptions about the same structure as that in the first embodiment will be omitted.

FIG. 8A is a top view of a modified MR element 15, thereby showing the magnetization free layer 60. FIG. 8B is a sectional view of the MR element 15, thereby showing the magnetization fixed layer 40, the nonmagnetic layer 50, and the magnetization free layer 60.

As shown in FIG. 8A, the modified MR element 15 is longitudinal in shape and has a longitudinal direction perpendicular to the lamination direction thereof (i.e., parallel to an in-plane direction of the modified MR element 15). As shown in FIG. 8A, the magnetization free layer 60 is a rectangle in shape when viewed from the top side, and the respective sides thereof are referred to as X and Y. In addition, Y is longer than X.

Thus, the magnetization free layer 60 is made to have a longitudinal shape, thereby resulting in magnetic shape anisotropy to make the magnetization of the magnetization free layer 60 in the longitudinal direction thereof. This arrangement reduces magnetostatic energy thereof.

As shown in FIG. 8C, the modified MR element 15 may be ellipsoidal in shape to have long and short axes when viewed from the top side. As described above also in this case, the magnetization free layer 60 makes the magnetization thereof in the direction of the long axis (the longitudinal direction). FIG. 8D is a sectional view of the modified MR element 15.

In this way, the magnetization of the magnetization free layer 60 can be weakly fixed in one direction. This allows it to set the magnetization direction of the magnetization free layer 60 different from the direction of the tensile stress applied to the modified MR element 15.

Although the rectangular and the ellipsoidal shapes have been illustrated in FIGS. 8A to 8D, any longitudinal shapes having a longitudinal direction can make the magnetization direction of the magnetization free layer 60 different from the direction of the tensile stress.

A method to manufacture the blood-pressure sensor 10 employing the modified MR element 15 according to this modification will be described below.

Materials of the substrate 20 include Si, glass, flexible plastic, soft magnetic metals. The substrate 20 is provided with high elasticity to be flexible, while the substrate 20 is provided with low stiffness to be indestructible. As a result, the high elasticity and low stiffness provide the substrate 20 with a high susceptibility to a pressure, thereby allowing it to acquire a large strain.

A substrate including Si, glass, or a soft magnetic metal becomes more flexible by thinning a portion of the substrate on which the MR element 15 is provided. A Si substrate is thinned by RIE (Reactive Ion Etching), i.e., selective etching after an MR element is provided thereon.

Firstly, a flexible plastic film is formed on a solid Si or a solid glass substrate by coating, vacuum depositions, or synthesis of plastic raw materials. Secondly, the MR element is formed on the flexible plastic film. Then, the flexible plastic film with the MR element formed thereon is detached from the solid substrate including Si or glass. Before the detaching, a fixture may be provided to support the flexible plastic film, thereby allowing it to easily handle a flexible substrate of the flexible plastic film at the subsequent manufacturing steps. Alternatively, a plastic film may be formed to be thick on the solid substrate so that the plastic film itself does not bend. Furthermore, the plastic film portion with the MR element formed thereon may be thinned so that the thinned plastic film portion becomes flexible.

Requirements to be met by a flexible plastic substrate will be described below. The first requirement relates to a water absorption rate and a vapor transmission rate. The water absorption and vapor transmission rates of Si or glass substrates are negligibly small whereas the rates of the plastic substrates cannot be neglected. The first reason why the rates of the plastic substrates cannot be neglected is that gases are released from a vacuum chamber. A substrate is mounted inside the vacuum chamber of a deposition system every time electrodes, an MR film etc. are formed to manufacture the MR element. The deposition system for the MR element operates at an atmospheric pressure of 10⁻⁹ Torr or less. It is, therefore, necessary to control the amount of gases released from the flexible plastic substrate. Bake-out of the flexible plastic substrate before being mounted inside the vacuum chamber is effective as well as the bake-out thereof in a preparation chamber having a baking heater before feeding the substrate to a deposition chamber. The second reason why the water absorption rate and vapor transmission rate of the plastic substrates cannot be neglected is that the substrate deforms. The large deformation of the substrate makes it impossible to form fine MR elements. Then, it is important to choose a material having the water absorption rate and vapor transmission rate which are as low as possible.

The second requirement to be met by the plastic substrate is a mechanical strength. The plastic substrate of the blood-pressure sensor desirably bends to flexibly follow contraction and expansion of a blood vessel. For this reason, highly elastic materials are employed which have preferably elastic modulus of 2 MPa to 15000 Mpa and more preferably 50 Mpa or higher. A tensile strength and a breaking elongation coefficient are taken into consideration as indexes of materials for the plastic substrate to guarantee that the materials do not break during usage. The tensile strength preferably ranges from 10 MPa to hundreds of MPa. The breaking elongation coefficient preferably ranges from 1% to 1000%, and is more preferably 400%.

The third requirement to be met by the plastic substrate is a heat resistance. The MR film needs to be subjected to the heat treatment in a magnetic field to have the magnetization of the magnetization fixed layer fixed in one direction. The plastic material needs to have such a heat-proof temperature as high as heat treatment temperatures. The rating index of the heat resistance is a linear expansion coefficient. The smaller the coefficient, the lower the thermal stress of the substrate. Heat treatments of about 300° C. are needed in the manufacturing process of the MR element. A substrate is needed which have a sufficiently small linear expansion coefficient so that even a temperature change of 300° C. brings about a small linear expansion to the substrate.

When the requirements mentioned above are taken into consideration, materials of the flexible plastic substrate preferably include polyimide and parylene.

A 500 nm thick aluminum oxide layer is formed as an insulating layer on the substrate 20 by sputtering.

Resist is applied onto the insulating layer by spin coating to be followed by lithographic patterning of the resist to remove a portion of the resist.

RIE removes a portion of the insulating layer from which the resist has been previously removed, thereby exposing a portion of the substrate 20 to the air.

The portion of the substrate 20 is provided with a laminated structure of Ta (5 nm)/Cu (400 nm)/Ta (20 nm) by sputtering using a mask to form the electrode 30. In addition, the values in the brackets denote the film thicknesses. The slash “/” denotes lamination and A/B/C shows that the “B” layer and the “C” layer are laminated on the “A” layer.

CMP (Chemical Mechanical Polishing) is employed to flatten the surface of the insulating layer, thereby exposing the electrode 30 on the surface of the insulating layer.

The MR film with a thickness of about 40 nm is formed by sputtering using a mask on the electrode 30 exposed on the surface of the insulating layer.

The MR film is fabricated using a mask so that two or more strips thereof are formed to have widths of 2 μm to 5 μm.

A silicon oxide layer with a thickness of about 200 nm is laminated on the insulating layer and the MR film.

Resist is applied onto the silicon oxide layer by spin coating. Then, the resist on the strips of the MR film is removed within a width of 1.5 μm to 5 μm in a direction perpendicular to the direction of the strips, thereby defining the shape of the MR film.

The silicon oxide layer on the area from which the resist has been previously removed as just mentioned above is removed with RIE and ion-milling, thereby exposing the surface of the MR film to the air.

The magnetization fixed layer 40 may be subjected to the heat treatment in a magnetic field after forming the MR element or just after forming the MR film in order to fix the magnetization thereof in one direction. When IrMn was employed for the antiferromagnetic layer, the magnetization fixed layer 40 was subjected to the heat treatment at 280° C. for 4 hours in a magnetic field of 7kOe.

An Au film with a thickness of about 100 nm is formed using a mask onto the surface of the MR film exposed on the surface of the silicon oxide layer to prepare the electrode 70, thereby manufacturing the blood-pressure sensor 10. After that, an Au pad is formed on the electrode 70.

### Modification 3

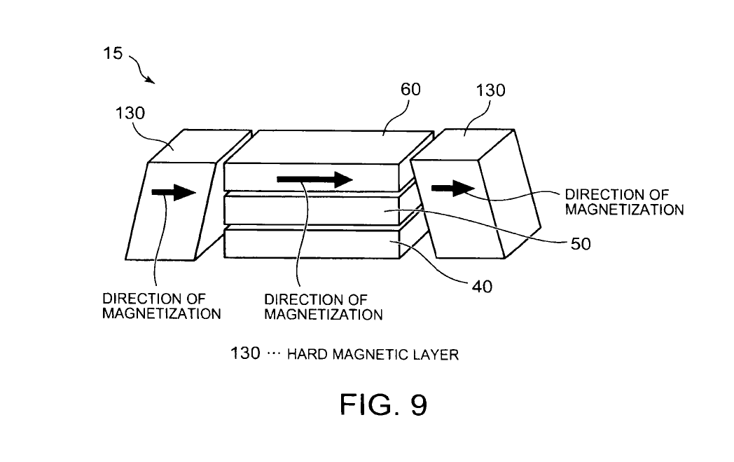

FIG. 9 is a view showing another modification of the MR element 15 according to the first embodiment. The electrodes are not shown in FIG. 9. Descriptions about the same compositions as those in the first embodiment will be omitted.

Hard magnetic layers 130 are provided so that a trilayer structure including the magnetization fixed layer 40, the nonmagnetic layer 50, and the magnetization free layer 60 are sandwiched between the two hard magnetic layers 130 in a direction perpendicular to the lamination direction of a modified MR element 15 over insulating layers not shown.

The magnetization of the hard magnetic layers 130 is set in one direction by means of annealing the hard magnetic layers 130 at not less than 200° C. and not more than 250° C. in a magnetic field of about 5kOe. The magnetic field generated from the hard magnetic layers 130 fixes the magnetization of the magnetization free layer 60 in the same direction as that of the magnetic field from the hard magnetic layers 130. Materials for the hard magnetic layer 130 include CoPt and FePt. The thickness of the hard magnetic layer 130 ranges from 5 nm to 20 nm, for example.

Next, a method to manufacture the blood-pressure sensor 10 using the MR element 15 according to this modification will be explained.

An about 500 nm thick aluminum oxide layer is formed on the substrate 20 by sputtering to provide an insulating layer.

Resist is applied onto the insulating layer by spin coating and then undergoes patterning by means of photolithography to remove a portion of the resist.

RIE removes a portion of the insulating layer from which the resist has been previously removed, thereby exposing a portion of the substrate 20 to the air.

The portion of the substrate 20 exposed on the surface of the insulating layer is provided with a laminated structure of Ta (5 nm)/Cu (400 nm)/Ta (20 nm) by sputtering using a mask to form the electrode 30.

CMP is employed to flatten the surface of the insulating layer, thereby exposing the electrode 30 on the surface of the insulating layer.

The MR film with a thickness of about 40 nm is formed by sputtering using a mask on the electrode 30 which is exposed on the surface of the insulating layer.

The hard magnetic layers 130 are formed on the side faces of the MR film over the insulating layer.

Next, an about 200 nm thick silicon oxide layer is laminated on the insulating layer, the MR film, and the hard magnetic layer by sputtering.

Resist is applied onto the silicon oxide layer by spin coating, and then a portion of the resist which is just above the MR film is removed.

RIE and ion-milling remove a portion of the silicon oxide layer from which the resist has been previously removed, thereby exposing a portion of the surface of the MR film to the air.

The portion of the surface of the MR film which is exposed on the silicon oxide layer is provided with a laminated structure of Ta (5 nm)/Cu (400 nm)/Ta (5 nm) using a mask to form the electrode 70, thereby manufacturing the blood-pressure sensor 10. After that, Au pads etc. are formed on the electrode 70.

The magnetization fixed layer 40 may undergo the heat treatment in a magnetic field after forming the MR element or just after forming the MR film in order to fix the magnetization thereof in one direction. When IrMn was employed for the antiferromagnetic layer, the magnetization fixed layer 40 was subjected to the heat treatment at 280° C. for 4 hours in a magnetic field of 7kOe.

### Modification 4

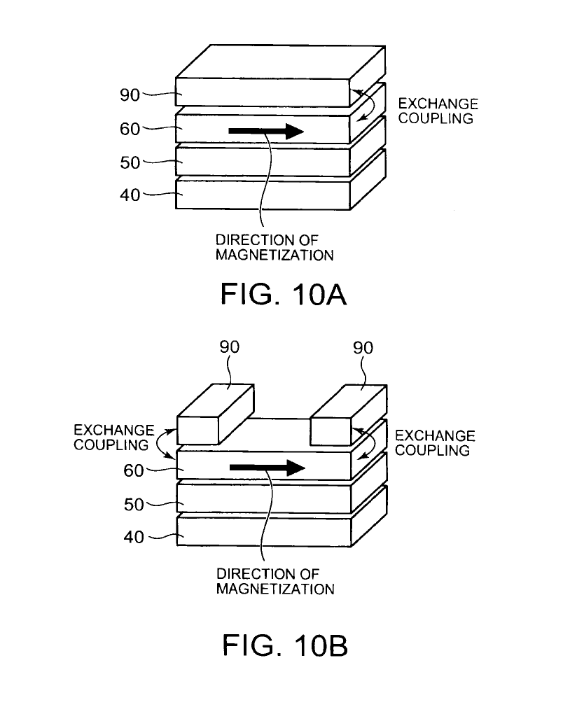

FIGS. 10A and 10B are views showing another two modifications of the MR element 15 according to the first embodiment. The electrodes are not shown. Descriptions about the same compositions as those in the first embodiment will be omitted.

The antiferromagnetic layer 90 is formed on the magnetization free layer 60. As shown in FIG. 10A, an IrMn layer with a thickness of not less than 1 nm and not more than 5 nm is provided on the magnetization free layer 60 as the antiferromagnetic layer 90. In this way, the antiferromagnetic layer 90 and the magnetization free layer 60 are weakly exchange-coupled to each other so that the magnetization of the magnetization free layer 60 is weakly fixed.

As shown in FIG. 10B, two antiferromagnetic layers 90 may be provided separately from each other on the magnetization free layer 60. An IrMn layer with a thickness of 5 nm to 7 nm is employed for the antiferromagnetic layer 90, for example. The antiferromagnetic layer 90 on the magnetization free layer 60 strongly couples the magnetization free layer 60 therewith. As a result, the magnetization of the magnetization free layer 60 in contact with the antiferromagnetic layer 90 is fixed in one direction. In FIG. 10B, the magnetization of the magnetization free layer 60 is fixed in one direction beneath the two antiferromagnetic layers 90. Therefore, the magnetization of the magnetization free layer 60 is drawn to rotate in the one direction even in other area of the magnetization free layer 60 on which the antiferromagnetic layers 90 are not provided.

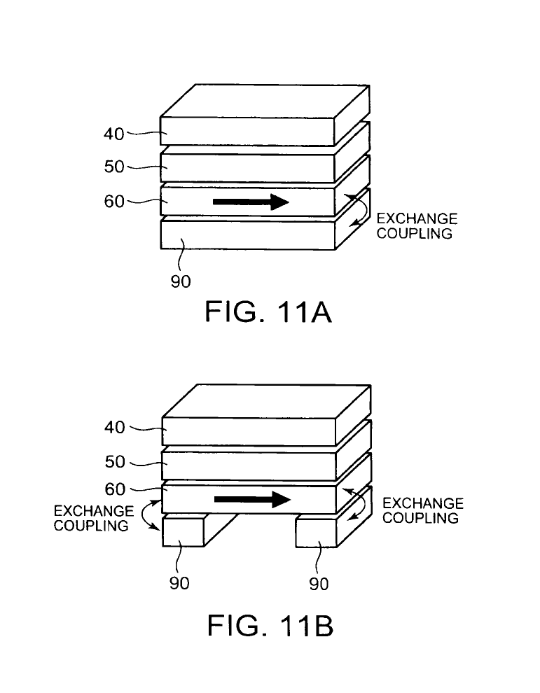

As shown in FIGS. 11A and 11B, the antiferromagnetic layer 90, the magnetization free layer 60, the nonmagnetic layer 50, and the magnetization fixed layer 40 may be laminated in this order.

According to this modification, it is possible to make the magnetization of the magnetization free layer 60 in one direction with comparatively low energy.

### Second Embodiment

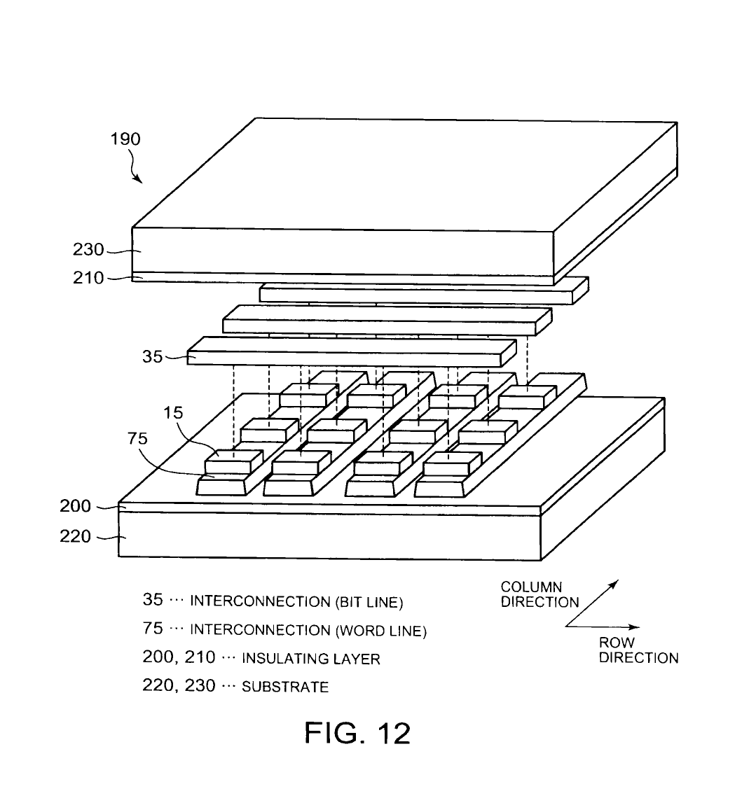

FIG. 12 is a view showing a blood-pressure sensor 190 according to a second embodiment. Descriptions about the same compositions as those in the first embodiment will be omitted. The blood-pressure sensor 190 employs two or more MR elements 15.

Interconnections 35 (referred to also as bit lines) are arranged in a row direction and interconnections 75 (referred to also as word lines) are arranged in a column direction. The MR elements 15 are provided at intersection points where the interconnections 35 and interconnections 75 intersect with one another. The MR elements 15 sandwiched between the interconnections 35 and interconnections 75 are further sandwiched between insulating layers 200 and 210. The insulating layers 200 and 210 are in contact with substrates 220 and 230, respectively.

The material of the interconnections 35 and 75 is the same as that of the electrodes 30 and 70. The MR element 15 does not need the electrodes 30 and 70.

The material of the substrates 220 and 230 are the same as that of the substrate 20.

Materials of the insulating layers 200, 210 include an aluminum oxide, e.g., Al₂O₃ and a silicon oxide, e.g., SiO₂.

When the substrates 220 and 230 are insulators, it is not necessary to employ the insulating layers 200 and 210. A soft magnetic layer may be inserted between the insulating layer 200 and the substrate 220 or between the insulating layer 210 and the substrate 230. The insertion of the soft magnetic layer allows it to reduce magnetic noises for the MR elements. Employing soft magnetic materials for the substrates 220, 230 can also reduce the magnetic noises.

An operation principle of the blood-pressure sensor 190 will be explained below.

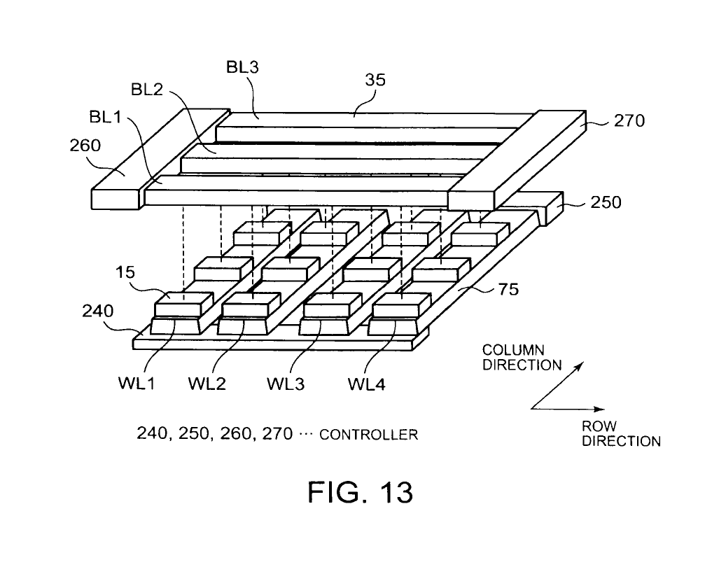

FIG. 13 is a view to explain the operation principle of the blood-pressure sensor 190.

Control units 240, 250, 260, and 270 are provided to the interconnections 35 and 75. The insulating layers 200, 210 and the substrates 220, 230 are not shown. The three interconnections 35 are illustrated and referred to as BL1, BL2, and BL3. The four interconnections 75 are illustrated and referred to as WL1, WL2, WL3, and WL4. The number of the interconnections 35 and 75 is not limited to these. It is assumed that a tensile stress acts on the blood-pressure sensor 190.

The control units 260 and 270 select BL1 from BL1 to BL3 to pass a current through BL1. When a current is passed through BL1, the control units 240 and 250 pass the current through each of the word lines from WL1 to WL4 in turn to measure the respective MR change rates of the MR elements arranged along BL1. The end of passing a current through WL4 is followed by selecting BL2 to pass a current through BL2. When a current is passed through BL2, the control units 240 and 250 again pass the current through each of the word lines from WL1 to WL4 in turn to measure the respective MR change rates of the MR elements arranged along BL2. In this way, the MR change rates are evaluated for all the MR elements 15 arranged between the interconnections 35 and 75 to be sent to CPU (Central Processing Unit, not shown), thereby allowing it to identify a specific MR element 15 having the largest MR change rate (referred to as “the largest MR element 15”). If the largest MR element 15 is identified, a blood pressure is measured therewith.

The above operation steps may be repeated at the same interval of time by minutes or hours. Data taken successively with the blood-pressure sensor 190 may be accumulated in a database connected to the blood-pressure sensor 190.

### Modification 5

FIG. 14 is a view showing a modification of the blood-pressure sensor 190 according to the second embodiment. Descriptions about the same compositions as those in the second embodiment will be omitted.

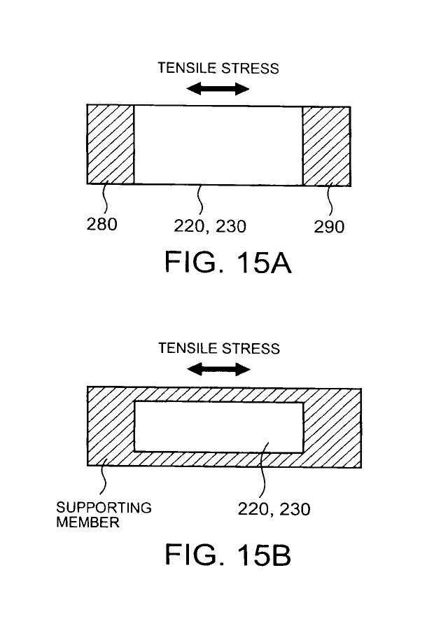

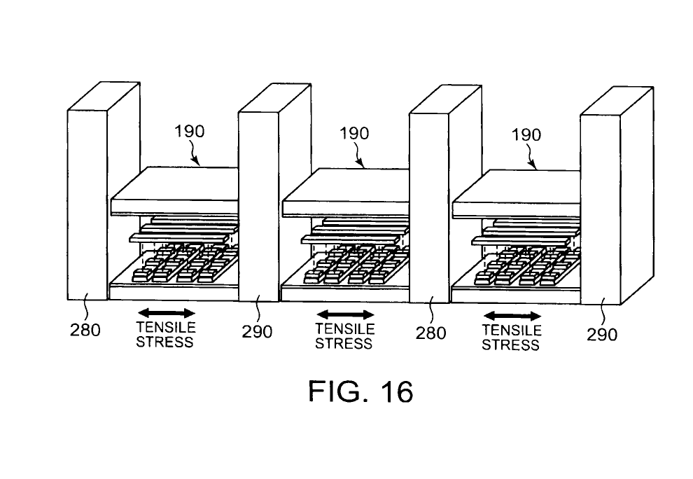

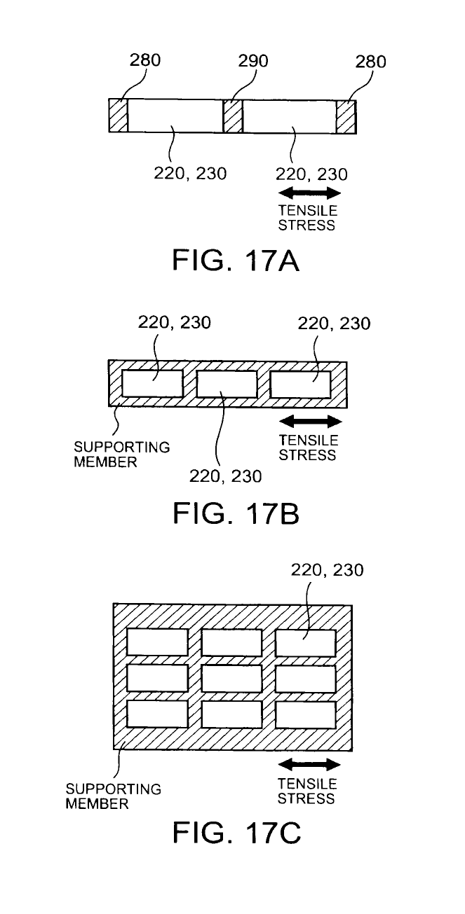

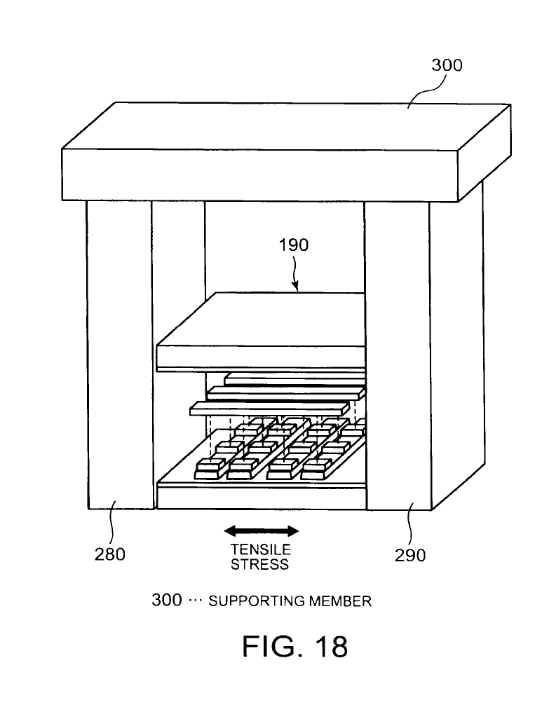

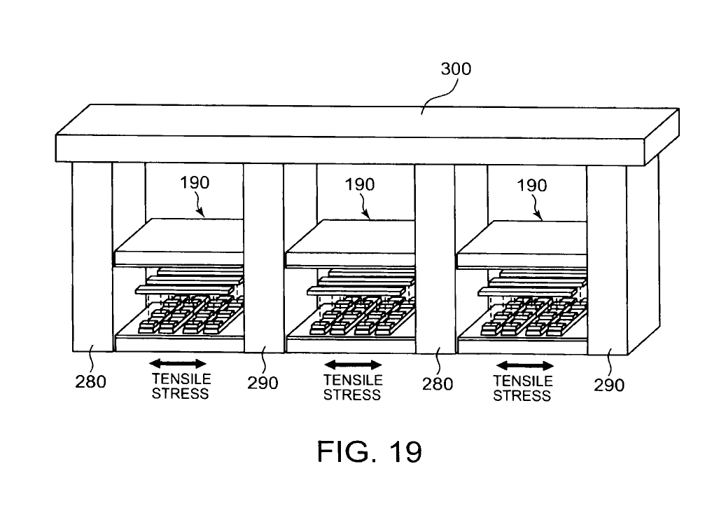

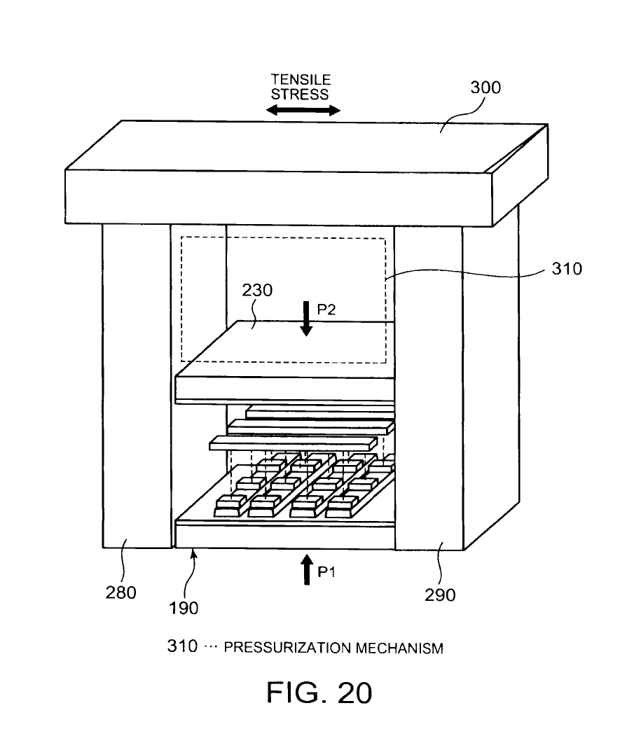

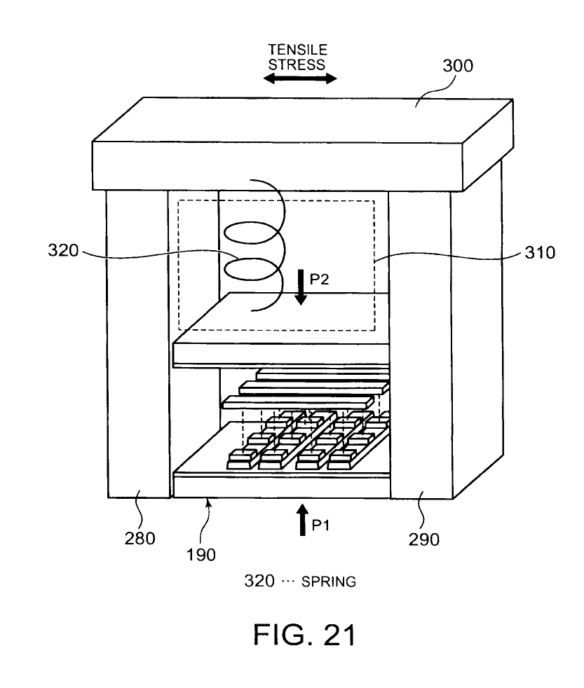

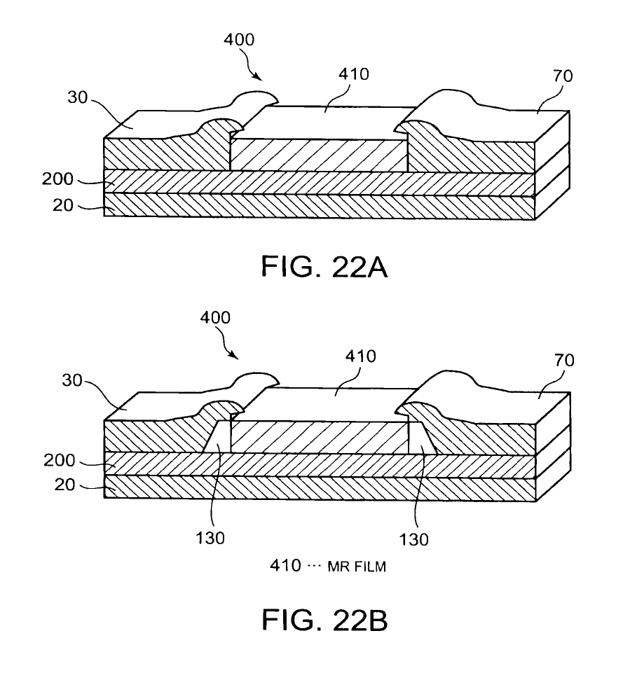

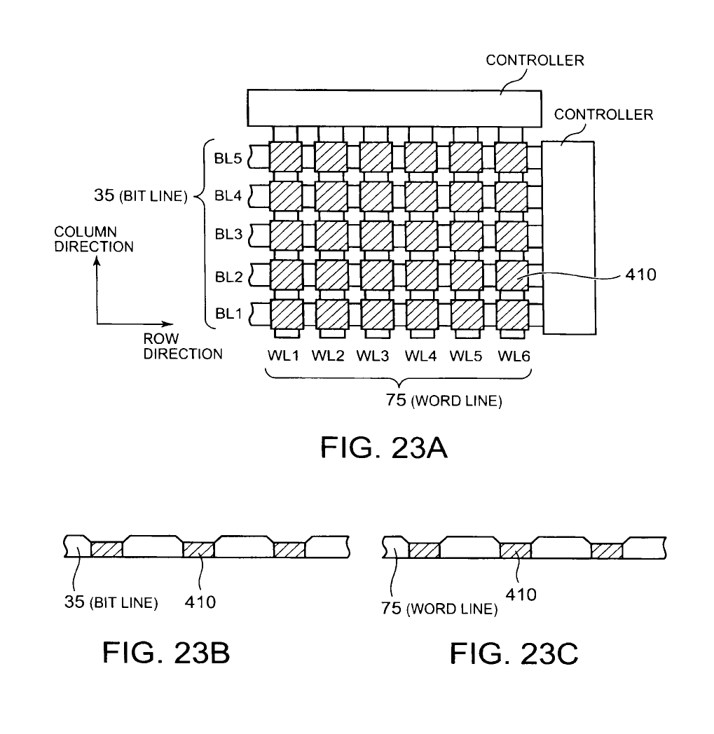

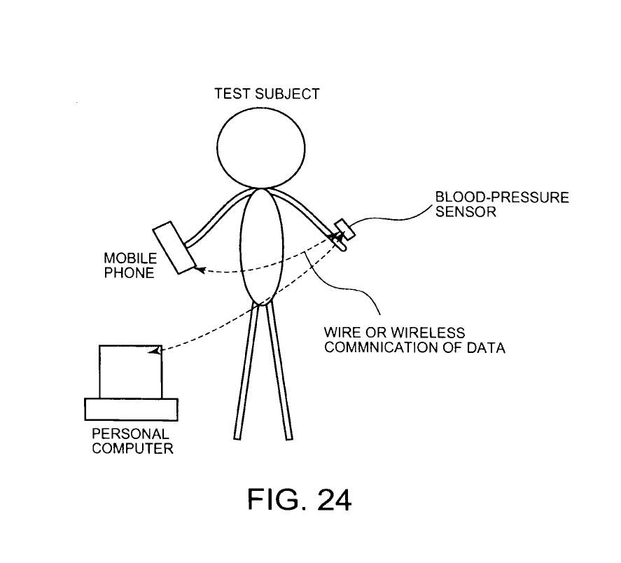

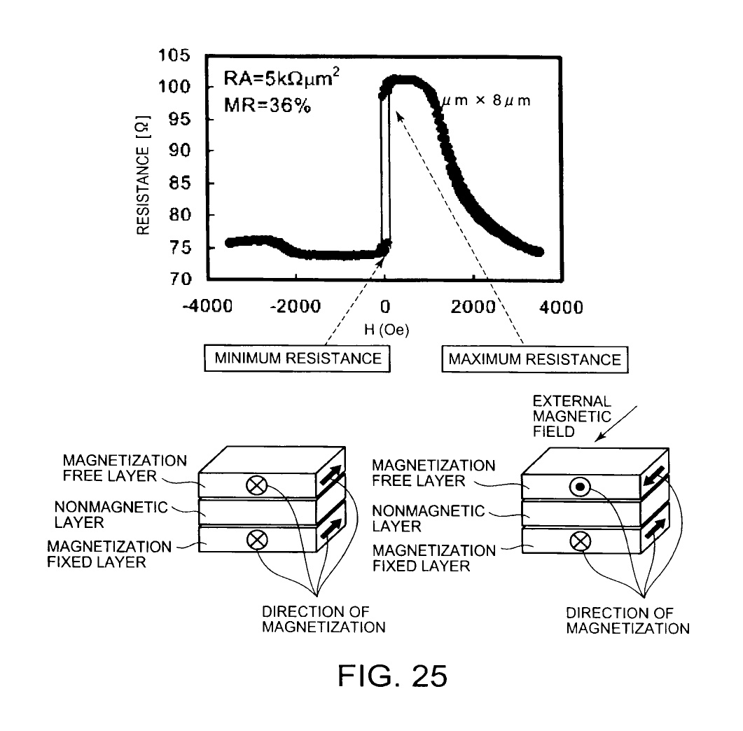

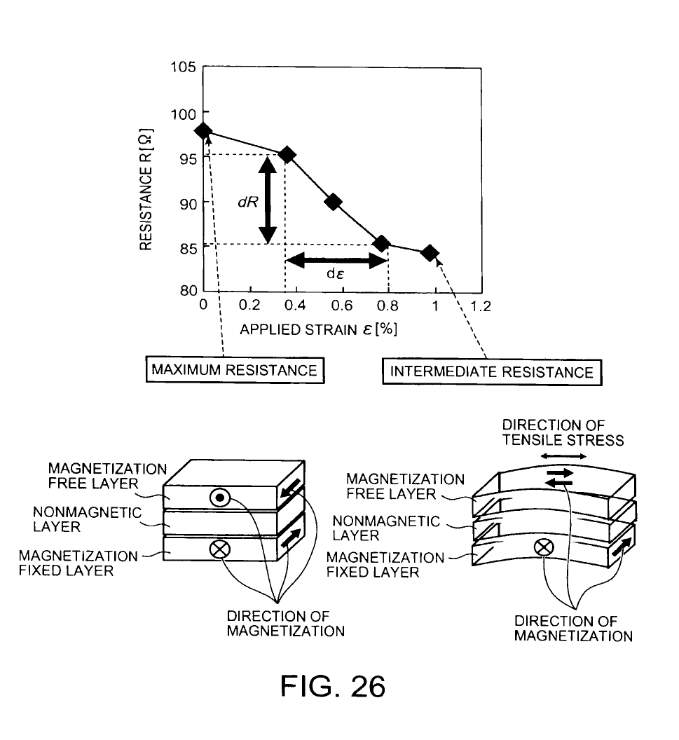

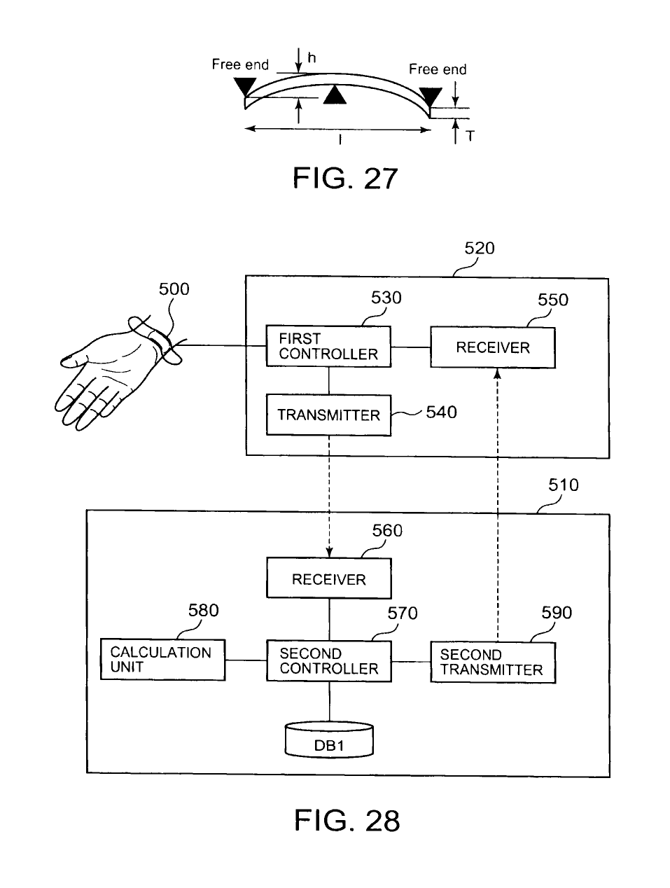

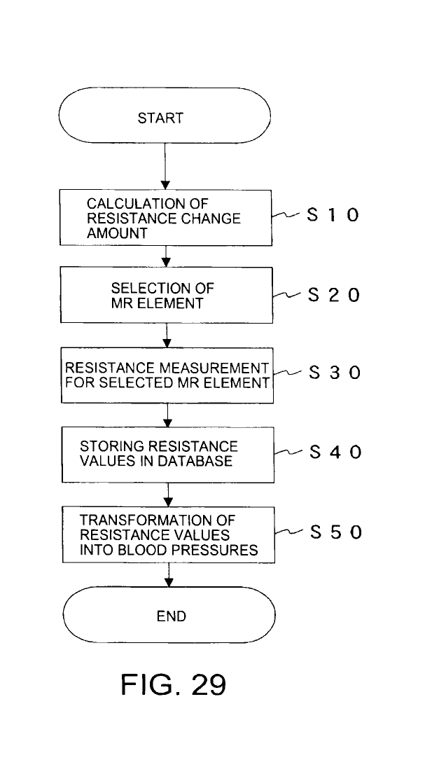

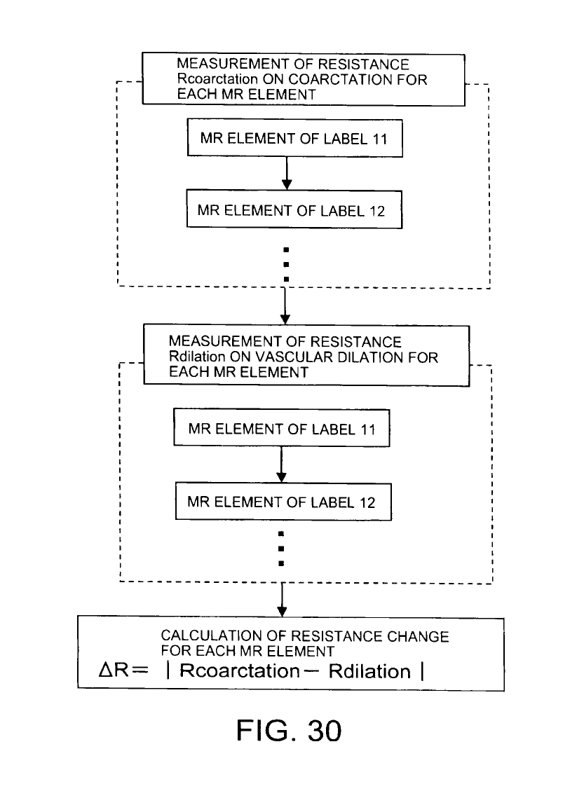

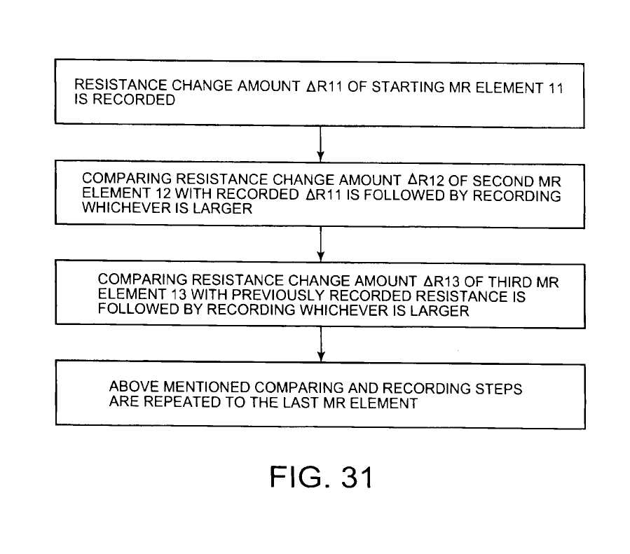

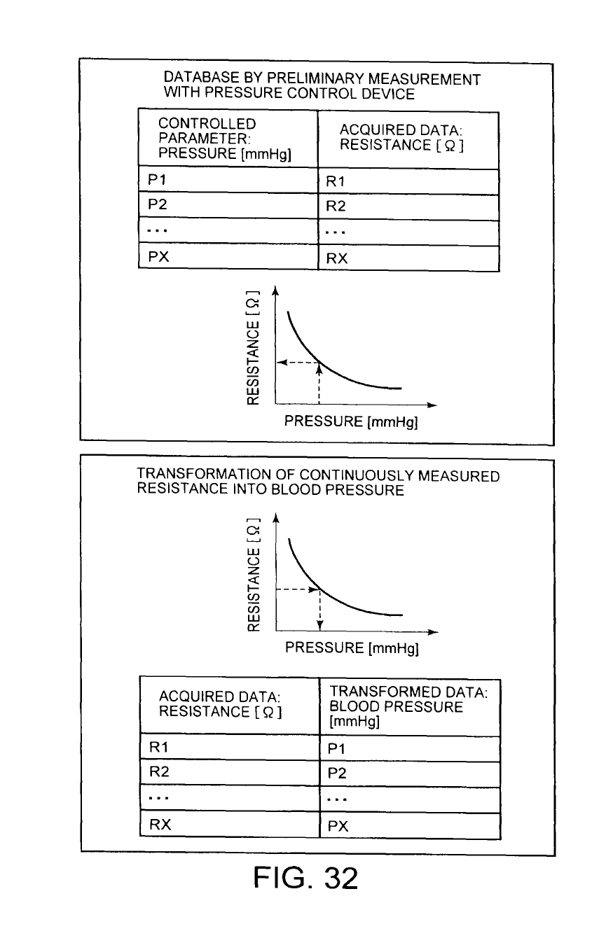

Both end faces of the substrates 220, 230 of the modified blood-pressure sensor 190 are in contact with supporting members 280, 290. In other words, both the end faces are sandwiched between the supporting members 280 and 290. The supporting members 280 and 290 face each other. The supporting members 280 and 290 are reference points for the substrates 220, 230 to strain in accordance with a tensile stress. In other words, the supporting members 280 and 290 serve as fixed ends. For this reason, a blood pressure can be measured more quantitatively. When the blood-pressure sensor 190 is viewed from an in-plane direction of the substrate 220 or the substrate 230, the blood-pressure sensor 190 is shown in FIG. 15A.

Materials of the supporting members 280 and 290 include Si or the like. The supporting members 280 and 290 are preferably plate-like in shape. The thickness thereof is about 1 μm, for example.

As shown in FIG. 15B, the supporting members may be provided to surround both the end faces of the substrates 220, 230. FIGS. 15A and 15B are views showing another two modifications of the blood-pressure sensor 190 according to the second embodiment.

When providing two or more blood-pressure sensors 190, the blood-pressure sensors 190 may be placed one by one in the respective gaps formed by two or more supporting members, e.g., as shown in FIG. 16. FIG. 17A is a view viewed from a direction perpendicular to the substrate 220 or 230 shown in FIG. 16 which is provided with interconnections 35 and 75.

As shown in FIG. 17B, the supporting members may be provided to surround both the end faces of the substrates 220, 230. When two or more blood-pressure sensors 190 are provided two-dimensionally as shown in FIG. 17C, the supporting members may be provided to surround both the end faces of the substrates 220, 230.

### Modification 6

FIG. 18 is a view showing another modification of the blood-pressure sensor 190 according to the second embodiment. Descriptions about the same compositions as those in the second embodiment will be omitted.

In addition to the supporting members 280 and 290 mentioned in the modification 5, another supporting member 300 is provided onto the end faces of the substrates 220, 230. Thus, forming the supporting member 300 allows it to fix the supporting members 280 and 290 more firmly. Therefore, a blood pressure can be measured more quantitatively.

When providing two or more blood-pressure sensors 190, the blood-pressure sensors 190 may be placed one by one in the respective gaps formed by two or more supporting members, e.g., as shown in FIG. 19.

### Modification 7

FIG. 20 is a view showing another modification of the blood-pressure sensor 190 according to the second embodiment. Descriptions about the same compositions as those in the second embodiment will be omitted.

A pressurization mechanism 310 is provided onto the substrate 230 included in the blood-pressure sensor 190. The pressure P2 of the pressurization mechanism 310 is preliminarily held in a range to balance the blood pressure P1 of a test subject therewith, thereby allowing it to measure a blood pressure more quantitatively. In this case, data of correlation between the pressures and the resistances outputted from the blood⁻pressure sensor 190 are preliminarily accumulated in order to obtain the absolute value of the blood pressure. Specifically, the pressure P1 is applied with a pressure generator for pressure control with varying the pressure P1 to acquire resistances R in response to the variation thereof. The data of correlation between the pressures P1 and the resistances R are used as a gauge for the blood-pressure sensor. When measuring actual blood pressures, the blood pressure sensor refers to the gauge previously accumulated from the data of resistances R for output of the blood pressure P1. The correlation between MR change rates and blood pressures can be measured using the pressurization mechanism 310.

The pressurization mechanism 310 is shown by the dashed line-enclosed area. The pressurization mechanism 310 can hold a constant pressure. The pressurization mechanism 310 may be arranged to be enclosed by the supporting members or to be set in a sealed housing which is provided on the substrate 230.

FIG. 21 is a view showing another modification of the blood-pressure sensor 190 according to the second embodiment. Alternatively, a spring 320 may be provided inside the pressurization mechanism 310 so that the pressure of the pressurization mechanism 310 is kept to be constant. A precision micro spring with a diameter of 800 μm can be employed for the spring 320, for example. Alternatively, two or more springs 320 may be provided.

Alternatively, two or more blood-pressure sensors 190 may be provided as shown in FIG. 19. In this case, springs having various spring constants are provided, thereby allowing it to measure blood pressures of various test subjects.

Alternatively, the pressure of the pressurization mechanism 310 may be electronically controlled from outside. For example, when using the sealed housing, the pressure of the pressurization mechanism 310 is electronically controlled to admit and release the air from outside.

### Third Embodiment

FIG. 22A is a view showing a blood-pressure sensor 400 according to a third embodiment. The third embodiment is different from the first and second embodiments in that the blood-pressure sensor 400 employs a CIP (Current in plane)-GMR effect. That is, a current is passed through the laminated film of the MR element in the in-plane direction thereof (in the direction perpendicular to the lamination direction thereof) to detect a MR change rate.

The blood-pressure sensor 400 is provided with an MR film 410 which is formed on an insulating layer 200 on the substrate 20, and the MR film 410 is sandwiched between a pair of the electrodes 30 and 70 in a direction perpendicular to the lamination direction thereof. When the substrate 20 is an insulator, the insulating layer 200 need not be provided.

The MR film 410 is the same as the MR element lacking the electrodes 30, 70. Therefore, descriptions thereof will be omitted.

FIG. 22B is a view showing another blood-pressure sensor 400 according to the third embodiment. As shown in FIG. 22B, the hard magnetic layers are provided between the electrode 30 and the MR film 410, and between the electrode 70 and the MR film 410.

### Modification 8

FIGS. 23A to 23C are views showing another modification of the blood-pressure sensor 400 according to the third embodiment. FIG. 23A is a view showing a circuit including the blood-pressure sensor 400. The substrate 20 and some elements are not shown. Moreover, descriptions about the same compositions as those in the third embodiment will be omitted.

As shown in FIG. 23A, two or more interconnections 35 are formed in the column direction, and two or more interconnections 75 are formed in the row directions, thereby forming a matrix with the interconnections 35 and 75. In the cross points of the interconnections 35 and the interconnections 75, each of the MR films 410 is provided between the interconnection 35 and the interconnection 75. The explanation of the operation principle will be omitted as it is the same as that was explained with reference to FIG. 13.

FIG. 23B is a view showing the blood-pressure sensor 400 viewed from the row direction. The MR films 410 are periodically buried in the respective interconnections 35 in the row direction.

FIG. 23C is a view showing the blood-pressure sensor 400 viewed from the column direction. The MR films 410 are periodically buried in the respective interconnections 75 in the column direction.

In addition, the above compositions differ from the second embodiment only in the current-flowing direction, thereby allowing it to employ the compositions for the modifications 5 to 7.

### Fourth Embodiment

FIG. 24 is a view showing a usage example of a blood-pressure sensor according to a fourth embodiment. FIG. 24 is a view showing a blood-pressure sensor to measure a blood pressure for a test subject. FIG. 24 also shows an example of the methods for power supply and data accumulation when the blood-pressure sensor is stuck on the blood-pressure measurement site. The blood-pressure sensors 10, 190, and 400 explained in the first, second, and third embodiments can be used as the blood-pressure sensor of this embodiment.

A small battery can also be employed for electric power supply. It is also possible to employ wireless electric power supply. As the data accumulation methods, data are wirelessly transmitted to be accumulated in the devices which include a mobile phone, a personal computer, and a wrist watch.

### EXAMPLE

A laminated structure of Al₂O₃ (20 nm)/Cu (400 nm for electrode)/IrMn (7 nm)/CoFe (3.4 nm)/Ru (0.8 nm)/FeCo (3 nm for magnetization fixed layer)/Al₂O₃ (1 nm for nonmagnetic layer)/FeCo (4 nm for magnetization free layer)/Cu (400 nm for electrode)/Ta (3 nm for protective layer) was prepared on a Si substrate using a sputtering method to form an MR element. Then, the MR element was processed to be a square with a side of 8 μm. The processed MR element was used as a TMR element.

FIG. 25 is a view showing a graph for the resistance measurements and magnetization arrangements of the processed MR element. The graph has the horizontal and vertical axes. The horizontal axis represents the magnetic field H (Oe) which was applied to the MR element to be swept from −4000Oe to +4000Oe. The vertical axis represents the resistances R (Ω) of the MR element at the respective points of the applied magnetic fields.

As shown in FIG. 25, the external magnetic field to be applied increases to result in a rapid increase in the resistance around at the original point. The minimum resistance shows that the magnetization directions of the magnetization free layer and the magnetization fixed layer are parallel to each other. The maximum resistance shows that the magnetization directions of the magnetization free layer and the magnetization fixed layer are antiparallel to each other. In the case shown in FIG. 25, the MR change rate, an areal resistance, and the magnetostriction constant of the magnetization free layer were 36%, 5 kΩμm², and 56 ppm, respectively. The areal resistance means a product of a cross section and a resistance. The cross section is cut perpendicularly to the lamination direction of the laminated films in the MR element. The resistance is measured between two electrodes through which a current is passed perpendicularly to the plane of the laminated film. The MR change rate means a value to be derived from the resistance change divided by the absolute value of the resistance. The magnetostriction constant λs shows an elongation quantity by which a ferromagnetic layer changes in length when an external magnetic field is applied to the ferromagnetic layer. The magnetostriction constant λs is expressed by the following equality, provided that a ferromagnetic body having the length “l” under no external magnetic field elongates by “Δl” under an external magnetic field.

λs=Δl/l

This phenomenon is called a magnetostriction effect. When the ferromagnetic layer elongates by Δl under the external magnetic field, the magnetization thereof is rotated in the direction in which the ferromagnetic layer elongates. This phenomenon is called an inverse magnetostriction effect. As described above, a strain is applied to give a tensile stress to the blood-pressure sensor, thereby elongating the magnetization free layer 60 to obtain the inverse magnetostriction effect. When the magnetostriction constant is negative, applying an external magnetic field to a magnetic layer results in compression of the magnetic layer.

As mentioned above, the MR element prepared was shown to output an excellent MR change rate in accordance with the strain.

FIG. 26 is a view showing a graph for the resistance measurements of the prepared MR element to which a tensile stress is applied and the magnetization arrangements of the MR element with and without the tensile stress. The vertical axis of the graph represents the resistances R (Ω) of the MR element. The horizontal axis thereof represents the strain (applied strain ε (permillage: ‰)). FIG. 27 is a view to explain how to strain a prepared MR element. The strain is given as shown in FIG. 27 with three points of the substrate fixed. Both the ends thereof are fixed and the middle point is pressed to produce a strain. Then, the strain ε is expressed with the following formula.

ε=6hT/l²

Here, “h”, “T”, and “l” represent a displacement in a direction perpendicular to the substrate surface, the thickness of the substrate, and a distance between the fixed ends, respectively.

The magnetization of the magnetization free layer is in the same direction as that of the magnetization fixed layer as a result of magnetic coupling therebetween when no strain is produced. In addition, the magnetization of the magnetization fixed layer was annealed at 280° C. in a magnetic field of 7kOe to be fixed after forming the MR film. The direction of the magnetic field for the annealing was parallel to an orientation flat of the substrate. Therefore, the magnetization direction of the magnetization free layer is the same as the orientation flat. These magnetization directions being memorized, the resistances were measure with giving a strain in a direction perpendicular to the magnetization directions. An external magnetic field of 6Oe is applied in a direction parallel to the magnetization direction of the magnetization fixed layer 40 during the measurement. In an actual blood-pressure sensor, a hard magnetic layer is arranged on the side wall of the MR element to apply an external magnetic field to the MR element, or an antiferromagnetic layer is made to be in contact with the magnetization free layer. The Si substrate was made to bend to strain the MR element so that a tensile stress was applied in a direction perpendicular to the magnetization direction of the magnetization free layer. The resistance of the MR element was measured with setting the applied strain ε to 0‰, 0.35‰, 0.55‰, 0.78‰, and 0.99‰.

FIG. 26 shows that the resistance changes in accordance with a change in the applied strain ε, thereby revealing that the MR element outputs an excellent MR change rate. Also is shown that the resistance decreases with increasing the applied strain. This comes from the following reason. That is, the magnetization directions of the magnetization fixed layer and the magnetization free layer are initially antiparallel to each other. Then, the magnetization of the magnetization free layer rotates to approach a direction parallel to the magnetization direction of the magnetization fixed layer.

A gauge factor is generally employed as an index of sensitivity to a strain. The gauge factor is defined as the MR change rate divided by a strain ε. The larger the gauge factor, the higher the sensitivity to the strain. This can be understood also in terms of the above definition of the gauge factor. In other words, the larger the MR change rate, the larger the gauge factor, provided that the strain ε is constant.

The prepared MR element had a gauge factor of 270. There is known a MEMS pressure sensor made of Si to have a gauge factor of about 140. The prepared MR element indicates a much larger gauge factor than the MEMS pressure sensor.

A laminated structure of Al₂O₃ (20 nm)/Cu (400 nm for electrode)/IrMn (7 nm)/CoFe (3.4 nm)/Ru (0.8 nm)/FeCoB (3 nm for magnetization fixed layer)/MgO (1 nm for nonnmagnetic layer)/FeCoB (4 nm for magnetization free layer)/Cu (400 nm for electrode)/Ta (3 nm for protective layer) was prepared on a Si substrate using a sputtering method to form an MR element. Then, the MR element was processed to be a square with a side of 8 μm. The MR element had an MR change rate of 200% and a gauge factor of 1000. Thus, the MR element is employed to allow it to enhance the gauge factor.

### Fifth Embodiment

FIG. 28 is a view showing a blood-pressure measurement system employing a blood-pressure sensor 500. The blood-pressure sensor 500 has the same composition as the blood-pressure sensors 190 and 400. The blood-pressure measurement site of a test subject is equipped with the blood-pressure sensor 500. Here, a wrist is illustrated as a blood-pressure measurement site. The blood-pressure measurement system according to this embodiment is assumed to be provided with the blood-pressure sensor 500 and an electronic device 510. Examples of the electronic device 510 include a TV set, a mobile phone, a hospital-use database, and a personal computer.

The blood-pressure sensor 500 is provided with a processor unit 520 therein.

The processor unit 520 includes a first controller 530, a transmitter 540 to transmit information from the first controller 530 to an outside, a second receiver 550 to receive information from the outside and transmit the information to the first controller 530.

In addition, “information” means data of blood pressures, resistance change rates, and resistance values.

The electronic device is provided with a receiver 560, a second controller 570, a calculation unit 580, a transmitter 590, and a database (referred to as DB1 below).

The receiver 560 receives information from the transmitter 540 to transmit the information to the second controller 570.

The second controller 570 transmits the information from the receiver 560 to the calculation unit 580 or the transmitter 590, or the second controller 570 stores the information in DB1.

The calculation unit 580 calculates the information from the second control unit 570. The calculation method will be mentioned later.

In addition, sending and receiving of information between the transmitter 540 and the receiver 560, and between the transmitter 590 and the receiver 550 are performed through wireless or wire communication.

FIG. 29 is a flow chart to illustrate operation steps of a blood-pressure measurement system using a blood-pressure sensor.

At Step S10, the first controller 530 instructs the blood-pressure sensor 500 to measure the resistance change amount of a blood-pressure measurement site. At this time, the resistance change amounts in all the MR elements provided to the blood-pressure sensor 500 are measured. The resistance change amounts which the blood-pressure sensor 500 has measured are transmitted as data to the receiver 560 of the electronic device 510 by the transmitter 540 through the first control unit 530. The data of the resistance change amounts received by the receiver 560 is transmitted to the calculation unit 580 through the second control unit 570. The calculation unit 580 calculates to transform the resistance change amounts into the absolute values thereof.

FIG. 30 is a view to show a measuring method of the resistance change amount of the blood-pressure sensor 500. The respective MR elements are selected, which are provided to the positions where the word lines and bit lines intersect with each other. For example, the MR element at the position where the word line WL1 and the bit line BL1 intersect with each other will be labeled “11” and the resistance thereat will be referred to as R11 below.

A current is passed through the respective MR elements arranged. For example, when N word lines and M bit lines are arranged over the blood-pressure sensor 500, a current is firstly passed through BL1 to BLM in sequence from WL1 to which a voltage is applied to supply the current to the respective bit lines. Secondly, the current is passed therethrough from WL2 in the same way. Thus, the same steps are repeated from WL1 to WLN. Furthermore, the steps is repeated on coarctation and vascular dilation in the same way. The resistances on coarctation and vascular dilation are referred to as Rcoarctation and Rdilation, respectively. Furthermore, the Rcoarctation and Rdilation of the MR element 11 are referred to as Rcoarctation11 and Rdilation11, respectively, in accordance with the label of the MR element. Next, the absolute value of resistance change amount on coarctation and vascular dilation in each MR element is calculated. That is, the formula ΔRXY=|RcoarctationXY−RdilationXY| is calculated for the MR element XY.

At Step S20, the calculation unit 580 identifies an MR element in terms of the position of the MR element allowing it to detect coarctation and vascular dilation as much as possible on the basis of the absolute value of resistance change amount.

FIG. 31 is a view to illustrate a selecting method of the MR element having the maximum absolute value of resistance change amount involved in coarctation and vascular dilation. That is, the resistance change amount ΔR11 of the MR element 11 and the resistance change amount ΔR12 of the MR element 12 are compared with each other, and whichever is larger is recorded. Subsequently, ΔR13 is compared with the recorded value, and whichever is larger is recorded. As above-mentioned comparing and recording steps are repeated to the last MR element MN to allow it to determine a specific MR element having the maximum absolute value of resistance change amount involved in coarctation and vascular dilation. After the specific MR element having the maximum absolute value of resistance change amount has been determined, the second control unit 570 instructs the receiver 550 to select the specific MR element via the transmitter 590. The receiver 550 transmits a piece of information of the instruction to the first control unit 530, and the first control unit 530 selects the MR element whose absolute value of the resistance change amount was maximum.

At Step S30, the first control unit 530 instructs to continuously acquire the resistance of the specific MR element selected at Step S20 via the blood-pressure sensor 500. Measuring for a prescribed period of time provides the maximum blood pressure, the minimum blood pressure, and a blood-pressure waveform. The prescribed period of time is exemplified on the second or minute time scale, e.g., 30 seconds or 2 minutes.

At Step S40, the resistance values acquired at Step 30 are stored in DB1 as a piece of data.

At Step S50, the resistance values which were continuously acquired in the former steps are transformed into blood pressures using the database of correlation between the previously acquired blood pressures and resistance values. When the database is created, a pressure is applied to the blood-pressure sensor using a pressure control device capable of controlling the same pressure as a blood pressure precisely. A pressure range includes a range from at least 50 mHg to 300 mmHg so that the range includes a blood pressure. The pressures to be measured for the database creation are acquired by 1 mmHg intervals, preferably by 0.01 mmHg intervals to provide blood-pressure measurements as precisely as possible. The data of resistance values corresponding to the blood pressures are acquired to create the database. FIG. 32 is a view to explain how the blood-pressure measurement system according to the fifth embodiment performs a transformation between resistance values and blood pressures. The database provides the correlation graph, e.g., as shown in the upper part of FIG. 32. As shown in the lower part of FIG. 32, the resistance values are inversely transformed into blood pressures in accordance with the database on measuring the blood pressures.

While certain embodiments of the invention have been described, these embodiments have been presented by way of example only, and are not intended to limit the scope of the inventions. Indeed, the novel elements and apparatuses described herein may be embodied in a variety of other forms; furthermore, various omissions, substitutions and changes in the form of the methods and systems described herein may be made without departing from the spirit of the invention. The accompanying claims and their equivalents are intended to cover such forms or modifications as would fall within the scope and spirit of the invention.

1. (canceled)

2. A MEMS pressure sensor comprising:
a substrate including
a first region, and
a second region that is located on an inner side of the first region and is flexible;
a magnetoresistive element including
a first electrode,
a second electrode disposed between the first electrode and the substrate, and
a laminated film disposed between the first electrode and the second electrode, wherein
the laminated film includes
a first magnetic layer having first magnetization,
a first magnetization free layer having second magnetization whose direction is variable, and
a non-magnetic layer disposed between the first magnetic layer and the first magnetization free layer.

3. The sensor of claim 2, further comprising
a supporting member configured to support the second region.

4. The sensor of claim 3, wherein the supporting member supports an entire periphery of the second region.

5. The sensor of claim 2, wherein the magnetoresistive element is disposed in a portion of the second region.

6. The sensor of claim 2, wherein the first magnetization free layer includes a multilayer film having a FeCo alloy magnetic layer.

7. The sensor of claim 2, wherein the first magnetization free layer includes a magnetic layer containing B.

8. The sensor of claim 2, wherein the first magnetization free layer has a shape having a longitudinal direction that is substantially perpendicular to a lamination direction in which the first magnetic layer, the non-magnetic layer, and the first magnetization free layer are laminated.

9. The sensor of claim 2, further comprising:
an antiferromagnetic layer that is disposed between the first electrode and the second electrode and on an opposite side to one side, where the non-magnetic layer is disposed, of the first magnetic layer or the first magnetization free layer.

10. The sensor of claim 2, further comprising:
a hard magnetic layer including a pair of CoPt alloy layers or a pair FePt alloy layers that sandwiches the laminated film therebetween in a direction substantially perpendicular to a lamination direction of the laminated film.

11. The sensor of claim 2, further comprising:
two antiferromagnetic layers that are disposed between the first electrode and the second electrode and on an opposite side to one side, where the non-magnetic layer is disposed, of the first magnetization free layer.

12. The sensor of claim 2, wherein the second region contains Si.

13. The sensor of claim 2, wherein
the second region includes an aluminum oxide layer, and
the second electrode contains a metal material.

14. The sensor of claim 2, wherein the number of magnetoresistive elements is plural.

15. The sensor of claim 14, wherein a pressure on an opposite side to one side, where the magnetoresistive elements are disposed, of the substrate is kept to be constant.

16. The sensor of claim 2, wherein a soft magnetic material is provided on the substrate aside from the magnetoresistive element.

17. The sensor of claim 2, further comprising:
a first insulating film disposed between the substrate and the magnetoresistive element;
a first soft magnetic layer disposed between the first insulating film and the substrate;
a second soft magnetic layer disposed on an opposite side to one side, where the laminated film is disposed, of the first electrode; and
a second insulating film disposed between the second soft magnetic layer and the first electrode.

18. A blood-pressure sensor comprising:
the MEMS pressure sensor of claim 2, wherein a blood pressure is measured with the MEMS pressure sensor.
